(19) United States
(12) Patent Application Publication
(10) Pub. No.: US 2021/0023074 A1
(43) Pub. Date: Jan. 28, 2021

(54) TREATMENT OF DEMYELINATING DISEASES

(71) Applicants: Victoria Link Ltd., Kelburn, Wellington (NZ); University of Kansas, Lawrence, KS (US)

(72) Inventors: Bronwyn Maree Kivell, Wellington (NZ); Anne Camille La Flamme, Wellington (NZ); Thomas Edward Prisinzano, Lawrence, KS (US)

(21) Appl. No.: 16/978,571

(22) PCT Filed: Mar. 7, 2019
(86) PCT No.: PCT/IB2019/051869

§ 371 Date: Sep. 4, 2020

(30) Foreign Application Priority Data
Mar. 8, 2018 (AU) 2018900753

(51) Int. Cl. A61K 31/485; A61P 25/28

(52) U.S. Cl. CPC A61K 31/485; A61P 25/28

(57) ABSTRACT

The present invention relates generally to methods of using nalmefene for treating and/or preventing demyelinating disease in a subject, and in particular for treating and/or preventing multiple sclerosis (MS). Also disclosed is nalmefene for use in treating and/or preventing MS as well as pharmaceutical compositions and unit dosage forms comprising nalmefene for use for treating and/or preventing demyelinating disease in a subject, and in particular for treating and/or preventing MS.

### 1. U.S. GOVERNMENT RIGHTS

This invention was made with government support under DA018151 awarded by the National Institutes of Health. The government has certain rights in the invention.

### 2. TECHNICAL FIELD

The invention relates generally to the use of nalmefene in the prevention and treatment of demyelinating diseases, in particular, multiple sclerosis.

### 3. BACKGROUND

The myelin sheath covers important nerve fibres in the central and peripheral nervous system of mammals, helping to facilitate transmission of neural impulses. Diseases that affect myelin interrupt these nerve transmissions. The developing myelin sheath can be affected by congenital metabolic disorders such as phenylketonuria, Tay-Sachs disease, Niemann-Pick disease, Hurler's syndrome, and Krabbe's disease. Demyelination can also occur in adults as a result of injury, metabolic disorders, immune attack, ischemia and toxic agents.

Demyelination impairs conduction of signals to the affected nerves, causing deficiency of sensation, movement, cognition and other functions. Demyelination of the central nervous system is associated with multiple sclerosis, Devic's disease, acute disseminated encephalomyelitis, adrenoleukodystrophy, leukoencephalopathy and Leber's optiv atrophy. Demyelination of the peripheral nervous symptom gives rise to diseases such as Guillain-Barre syndrome, chronic inflammatory demyelinating polyneuropathy, Charcot Marie Tooth (CMT) disease and progressing inflammatory neuropathy.

Multiple sclerosis (MS) is the most well-known demyelination disease, affecting about 2.5 million people worldwide. Sufferers endure a range of symptoms including fatigue, vision problems, numbness, cognitive impairment, incontinence, poor balance and muscle weakness, ultimately leading to paralysis. MS can follow four major disease courses, each of which can be mild, moderate or severe:
1. Relapsing-Remitting MS (RRMS)—clearly defined attacks (flare-ups) of worsening neurological function followed by partial or complete remission
2. Primary-Progressive MS (PPMS)—slowly worsening neurological function at variable rates, with no distinct remission
3. Secondary-Progressing MS (SPMS)—an initial period of RRMS is followed by a steady worsening, with or without flare-ups and remissions
4. Progressive-Relapsing MS (PRMS)—steadily worsening neurological function with clear flare-ups and partial or no remission.

While there is no cure for MS, many FDA approved drugs such as beta-interferon and glatiramer acetate are used to reduce relapse rates and the formation of new lesions. Unfortunately, current treatments are not very successful in preventing the disability associated with MS and are more successful in treating RRMS than other types. The inability of current drugs to stop or reverse disease progression and disability are largely due to their failure to support remyelination. Clearly, alternative treatments are needed; particularly treatments are able to treat the demyelination.

It is therefore an object of the present invention to go some way towards meeting this need in the art, to provide products and methods useful in the treatment of demyelinating diseases, and in particular to provide products and methods that are useful for stopping and/or reversing disease progression due to demyelination, including disease progression due to MS, and/or to at least to provide the public with a useful choice.

### 4. SUMMARY OF THE INVENTION

In one aspect the invention provides a pharmaceutical composition comprising nalmefene (NaIM) and pharmaceutically acceptable excipients for treating a demyelinating disease in a subject in need thereof.

In another aspect the invention provides a pharmaceutical composition comprising nalmefene and at least one pharmaceutically acceptable carrier or excipient for use for treating a demyelinating disease in a subject in need thereof.

In another aspect the invention provides unit dosage forms comprising about 0.01 to about 10 mg nalmefene and at least one pharmaceutically acceptable carrier or excipient. In one embodiment a unit dosage form comprises about 0.01 to about 2.0 mg of nalmefene and at least one pharmaceutically acceptable carrier or excipient. In one embodiment a unit dosage form comprises about 0.05 to about 0.6 mg nalmefene and at least one pharmaceutically acceptable carrier or excipient.

In one aspect the invention provides unit dosage forms comprising about 0.01 to about 10 mg nalmefene, about 0.05 to about 5 mg, about 0.1 to about 1 mg, about 0.2 to about 0.9 mg, about 0.3 to about 0.8 mg, about 0.4 to about 0.7, about 0.5 mg to about 0.6 mg, or about 0.6 mg nalmefene, and at least one pharmaceutically acceptable carrier or excipient.

In another embodiment, the unit dosage form is for treating a demyelinating disease in a subject in need thereof, preferably MS. In another embodiment, the unit dosage form is for oral administration.

In another aspect the invention provides a method of treating a demyelinating disease in a subject in need thereof, comprising administering a therapeutically effective amount of nalmefene to the subject.

In another aspect the invention provides a method of treating a demyelinating disease in a subject comprising identifying a subject who would benefit from a decreased level of demyelination and administering to the subject a therapeutically effective amount of an agent that decreases the level of demyelination in the subject relative to the level of demyelination before administering the agent, wherein the agent comprises nalmefene.

In another aspect the invention provides a method of treating a demyelinating disease in a subject in need thereof, comprising administering to the subject a therapeutically effective amount of an agent that decreases the level of demyelination in the subject relative to the level of demyelination before administering the agent, wherein the agent comprises nalmefene.

In another aspect the invention provides a method of increasing remyelination in a subject in need thereof, comprising administering a therapeutically effective amount of nalmefene to the subject.

In another aspect the invention provides a method of increasing remyelination in a subject comprising identifying a subject who would benefit from an increased level of remyelination and administering to the subject a therapeutically effective amount of an agent that increases the level of remyelination in the subject relative to the level of remyelination before administering the agent, wherein the agent comprises nalmefene.

In another aspect the invention provides a method of increasing remyelination in a subject in need thereof, comprising administering to the subject a therapeutically effective amount of an agent that increases the level of remyelination in the subject relative to the level of remyelination before administering the agent, wherein the agent comprises nalmefene.

The invention also provides a use of nalmefene in the manufacture of a medicament for treating a demyelinating disease in a subject in need thereof.

The invention also provides a use of nalmefene in the manufacture of a medicament for increasing remyelination in a subject in need thereof.

The invention also provides nalmefene for use for treating a demyelinating disease.

In one embodiment the disease is a demyelinating myelinoclastic disease.

In one embodiment the disease is a demyelinating leukodystrophic disease.

In one embodiment the demyelinating disease is a central nervous system demyelinating disease. In one embodiment the central nervous system demyelinating disease is selected from the group comprising MS (including clinically isolated syndrome; CIS), optic neuritis, Devic's disease, inflammatory demyelinating diseases, central nervous system neuropathies, myelopathies like Tabes dorsalis, leukoencephalopathies, leukodystrophies, or a combination thereof.

In one embodiment the demyelinating disease is MS.

In another embodiment the demyelinating disease is a peripheral nervous system demyelinating disease. In one embodiment the peripheral nervous system demyelinating disease is elected from the group comprising Guillain-Barre syndrome and its chronic counterpart, chronic inflammatory demyelinating polyneuropathy, anti-myelin associated glycoprotein (MAG) peripheral neuropathy, Charcot Marie Tooth (CMT) disease, copper deficiency and progressive inflammatory neuropathy.

In another aspect the invention provides a method of attenuating demyelination in a subject in need thereof, comprising administering a therapeutically effective amount of nalmefene to the subject and thereby attenuating a level of demyelination in the subject relative to the level of demyelination when nalmefene is not administered.

The invention also provides a use of nalmefene in the manufacture of a medicament for attenuating demyelination in a subject in need thereof. In one embodiment, the subject is a human with MS.

In another aspect the invention provides a method of treating MS in a subject in need thereof, comprising administering a therapeutically effective amount of nalmefene to the subject.

In another aspect the invention provides a method of treating MS in a subject in need thereof, comprising administering to the subject a therapeutically effective amount of an agent that decreases a level of demyelination in the subject relative to the level before administering the agent and/or that increases a level of remyelination in the subject in the subject relative to the level before administering the agent, wherein the agent comprises nalmefene.

The invention also provides a use of nalmefene in the manufacture of a medicament for treating MS in a subject in need thereof.

The invention also provides nalmefene for use for treating MS in a subject in need thereof.

In one embodiment the subject has RRMS. In one embodiment the subject has PPMS. In one embodiment the subject has, or is diagnosed as having, SPMS. In one embodiment the subject has, or is diagnosed as having, PRMS. In one embodiment the subject has, or is diagnosed as having, Clinically Isolated Syndrome (CIS).

In one embodiment the treatment of MS results in one or more clinical outcomes when compared to subjects not treated with nalmefene selected from the group consisting of:
(a) a decrease in MS disease progression;
(b) a decrease in MS disease severity;
(c) a decrease in nerve cell demyelination;
(d) a decrease in frequency or severity of relapsing MS attacks;
(e) a decrease in MS clinical symptoms;
(f) the healing of damaged nerve tissue (neuro-restoration);
(g) an increase in remyelination of demyelinated nerves in the central nervous system (neuro-restoration/protection);
(h) the protection of damaged nerve tissue from further disease activity (neuroprotection);
(i) the promotion neuronal outgrowth (neuro-regeneration) in the central nervous system;
(j) a decrease in disability caused by MS;
(k) an improvement of nerve function; and
(l) an enhanced rate of remission.

In another embodiment the treatment of MS results in a reduction of one or more clinical symptoms of MS including, but not limited to loss of sensitivity or changes in sensation such as tingling, pins and needles or numbness, muscle weakness of variable severity, very pronounced reflexes, muscle spasms, or difficulty in moving; difficulties with coordination and balance (ataxia); spasticity; problems with speech or swallowing, visual problems (nystagmus, optic neuritis or double vision), fatigue, acute or chronic pain, facial pain (trigeminal neuralgia), bladder and bowel difficulties, incontinence, reduced cognitive ability, depression, anxiety and other emotional abnormalities, sexual dysfunction, Uhthoff's phenomenon (a worsening of symptoms due to exposure to higher than usual temperatures), and Lhermitte's sign (an electrical sensation that runs down the back when bending the neck).

In one aspect the invention provides a method of accelerating remission from MS in a subject in need thereof, the method comprising administering a therapeutically effective amount of nalmefene to the subject.

In one aspect the invention provides a method of accelerating remission from MS in a subject in need thereof, the method comprising administering a therapeutically effective amount of an agent that decreases the level of demyelination in the subject relative to the level of demyelination before administering the agent, wherein the agent comprises nalmefene.

In one aspect the invention provides a method of accelerating remission from MS in a subject in need thereof, the method comprising administering a therapeutically effective amount of an agent that increases the level of remyelination in the subject relative to the level of remyelination before administering the agent, wherein the agent comprises nalmefene.

The invention also provides a use of nalmefene in the manufacture of a medicament for accelerating remission from MS in a subject in need thereof.

The invention also provides nalmefene for use for accelerating remission from MS in a subject in need thereof.

In another aspect the invention relates to a method of treating a demyelinating disease in a subject comprising identifying a subject who would benefit from a decreased level of demyelination and administering to the subject a therapeutically effective amount of an agent that decreases the level of demyelination relative to the level of demyelination before administering the agent, wherein the agent comprises nalmefene.

In another aspect the invention relates to a method of increasing remyelination in a subject comprising identifying a subject who would benefit from an increased level of remyelination and administering to the subject a therapeutically effective amount of an agent that increases the level of remyelination relative to the level of remyelination before administering the agent, wherein the agent comprises nalmefene.

In the above methods of the invention:

In one embodiment the therapeutically effective amount for a subject is equivalent to a dose of about 0.003 to about 0.3 mg/kg in mice.

In one embodiment the subject is human. In one embodiment the method comprises administering about 0.01 to about 10 mg nalmefene to the subject daily, 0.01 to about 2.0 mg nalmefene to the subject daily, or about 0.05 to about 0.6 mg nalmefene to the subject daily.

In another embodiment the method comprises administering about 0.01 to about 10 mg nalmefene, about 0.05 to about 5 mg, about 0.1 to about 1 mg, about 0.2 to about 0.9 mg, about 0.3 to about 0.8 mg, about 0.4 to about 0.7, about 0.5 mg to about 0.6 mg, or about 0.6 mg nalmefene daily.

In some embodiments the method comprises a long duration therapy.

In some embodiments the long duration therapy comprises administration of a therapeutically effective dose of nalmefene to a subject in need thereof for at least 5 days, at least 6 days, or at least 7 days.

In some embodiments a long duration therapy comprises administration of a therapeutically effective dose of nalmefene to a subject in need thereof for at least a week, at least 2 weeks, at least 3 weeks, at least 4 weeks, at least 6 weeks, or at least 8 weeks.

In some embodiments the long duration therapy comprises administration for at least 5 days, at least 6 days, at least 7 days, at least 14 days, at least 21 days, at least 28 days, at least 35 days, at least 42 days, at least 45 days, at least 60 days, at least 120 days, at least 240 days, or at least 360 days.

In some embodiments the long duration therapy comprises a dosing gap of at least 1 day.

Other aspects of the invention may become apparent from the following description which is given by way of example only and with reference to the accompanying figures.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art. However, these external documents and references are all cited herein by reference in their entireties or at least to the extent described herein.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

### 5. BRIEF DESCRIPTION OF THE DRAWINGS

The invention will now be described by way of example only and with reference to the drawings in which:

FIG. 1 is a graph showing the progression of disease in mice which have experimental autoimmune encephalomyelitis (EAE) over 45 days, wherein the mice in Example 1 were treated with 0.01, 0.03, or 0.1 mg/kg nalmefene daily from onset (day 17).

FIG. 2 is two graphs showing the total disability of EAE mice over (A) 45 days and (B) 18 days wherein the mice in Example 2 were treated with 0.03 or 0.1 mg/kg nalmefene daily from onset (day 17).

FIG. 3 is a graph showing the % weight change of EAE mice in Example 3 over 45 days wherein the mice were treated with 0.03 or 0.1 mg/kg nalmefene daily from onset (day 17).

FIG. 4 is three graphs showing immune cell infiltration into the brain of EAE mice in Example 4 after 45 days, wherein the mice were treated with 0.03 or 0.1 mg/kg nalmefene daily from onset (day 17).

FIG. 5 is a graph showing the progression of disease in EAE mice in Example 5 over 45 days, wherein the mice, which had not yet developed EAE, were treated with 0.03 or 0.1 mg/kg nalmefene daily from onset (day 17).

FIG. 6 is a series of Transmission Electron Microscope (TEM) images of spinal cord sections from EAE mice in Example 6 after 45 days, wherein the mice were treated with 0.03 mg/kg nalmefene daily from onset (day 17).

FIG. 7 shows that nalmefene promotes functional recovery from paralysis when administered therapeutically (at disease onset) in the EAE model of MS.

FIG. 8 shows that nalmefene modestly alters peak disease when administered therapeutically in the EAE model of MS.

FIG. 9 shows that nalmefene promotes full recovery from EAE-induced paralysis when administered therapeutically.

FIG. 10 shows that nalmefene promotes sustained recovery from EAE-induced paralysis when administered therapeutically.

FIG. 11 shows that nalmefene reduces the immune cell infiltration into the brain when administered therapeutically in the EAE model of MS.

FIG. 12 shows that myelination is improved in mice treated with nalmefene after the onset of paralysis in the EAE model of MS.

FIG. 13 shows that nalmefene does not alter the proportion of major lymphocyte populations in the spleen during the chronic phase of EAE.

FIG. 14 shows that nalmefene does not alter the number of CD4 T helper cells in the spleen, but rather shifts the CD4 T cells from an effector to memory phenotype suggestive of immune resolution during the chronic phase of EAE.

FIG. 15 shows that nalmefene reduces disease but does not enable full recovery when the kappa opioid receptor (KOR) is blocked. Additionally, blocking the mu opioid receptor (MOR) with BFNA, a MOR antagonist, does not enable significant and sustained recovery from paralysis.

FIG. 16 shows that activation of the KOR is required but blocking the activity of the MOR is not required for full recovery from paralysis mediated by nalmefene.

FIG. 17 shows that nalmefene is more effective at promoting functional recovery than clemastine fumarate, a known remyelinating drug.

FIG. 18 shows that nalmefene promotes a greater and more sustained recovery than clemastine fumarate, a known remyelinating drug.

FIG. 19 shows that nalmefene treatment enhances remyelination when administered therapeutically in the cuprizone demyelination disease model of MS.

### 6. DETAILED DESCRIPTION OF THE INVENTION

### 6.1 Nalmefene

Nalmefene is a drug commonly prescribed for management of alcohol dependence. It is a non-narcotic opioid with μ-opioid receptor (MOR) antagonist activity and partial x-opioid receptor (KOR) agonist activity. The inventors have now found that nalmefene is a surprisingly effective treatment for demyelination diseases.

The generic name “nalmefene” refers to the compound:

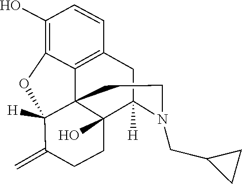

The IUPAC name for nalmefene is (4R,4aS,7aS,12bS)-3-(cyclopropylmethyl)-7-methylidene-2,4,5,6,7a,13-hexahydro-1H-4,12-methanobenzofuro[3,2-e]isoquinoline-4a,9-diol. Its CAS number is 58895-64-0. It is also known as ((5a)17-(cyclopropylmethyl)-4,5-epoxy-6-methylenemorphinan-3,14-diol. Nalmefene HCl may also be referred to by brand names Revex and Selincro TRK.

As used herein the term “nalmefene” refers to the compound identified above as well as to its pharmaceutically acceptable salts, prodrugs and solvates.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, ethanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids.

The term “solvate” refers to an aggregate that consists of a solute ion or molecule with one or more solvent molecules. “Solvates” include hydrates, that is, aggregates of a compound of interest with water.

The term “prodrug” refers to a drug that is administered in an inactive or significantly less active form which, once administered, is metabolized in vivo into its active parent form. Prodrugs of nalmefene include its alkyl esters, for example, palmitate, octadecyl glutarate and decyl carbamate esters of nalmefene.

Nalmefene can be purchased from small molecule suppliers such as Med Chem Express, Monmouth Junction and New Jersey, USA; AdooQ BioScience, Irvine Calif., USA.

### 6.2 Pharmaceutical Compositions of Nalmefene

There is a lack of effective treatments for demyelinating diseases, including MS, and in particular, there are few effective agents that act to reduce demyelination and/or to increase remyelination. Surprisingly, the inventors have found that pharmaceutical compositions containing nalmefene can be used to treat demyelination diseases including but not limited to MS by acting to increase remyelination and/or to decrease demyelination.

Accordingly, in one aspect the invention relates to a pharmaceutical composition comprising nalmefene and pharmaceutically acceptable excipients for treating a demyelinating disease and/or for increasing remyelination in a subject in need thereof.

In another aspect the invention provides a pharmaceutical composition comprising nalmefene and at least one pharmaceutically acceptable carrier or excipient for use for treating a demyelinating disease in a subject in need thereof.

This term “pharmaceutical composition” as used herein encompasses a product comprising one or more active agents, and pharmaceutically acceptable excipients comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by bringing the active agent into association with a liquid carrier, a finely divided solid carrier or both, and then, if necessary, shaping the product into the desired formulation. Said compositions are prepared according to conventional mixing, granulating, or coating methods, respectively, and contain a percentage (%) of the active ingredient as can be determined by a skilled worker in view of the art.

The term “comprising” as used herein means “consisting at least in part of”. When interpreting each statement in this specification that includes the term “comprising”, features other than that or those prefaced by the term may also be present. Related terms such as “comprise” and “comprises” are to be interpreted in the same manner.

The term “consisting essentially of” as used herein means the specified materials or steps and those that do not materially affect the basic and novel characteristic(s) of the claimed invention.

The term “consisting of” as used herein means the specified materials or steps of the claimed invention, excluding any element, step, or ingredient not specified in the claim.

By “pharmaceutically acceptable excipient” or “pharmaceutically acceptable carrier” it is meant that the excipient must be compatible with the other ingredients of the formulation and not harmful to the subject to whom the composition is administered.

Pharmaceutical compositions of the invention may be administered topically, orally or parenterally.

For example, the pharmaceutical compositions may be administered orally, including sublingually, in the form of capsules, tablets, elixirs, solutions, suspensions, or boluses formulated to dissolve in, for example, the colon or duodenum. The formulations may comprise excipients such as starch or lactose or flavouring, preserving or colouring agents.

The pharmaceutical compositions may be injected parenterally, for example, intravenously, intramuscularly or subcutaneously. For parenteral administration, the compositions may be formulated in a sterile aqueous solution or suspension that optionally comprises other substances, such as salt or glucose.

The compositions may be administered topically, in the form of sterile creams, gels, pour-on or spot-on formulations, suspensions, lotions, ointments, dusting powders, drug-incorporated dressings, shampoos, collars or transdermal patches. For example, the compositions of the invention may be incorporated into a cream comprising an aqueous or oily emulsion of polyethylene glycols or liquid paraffin; an ointment comprising a white wax soft paraffin base; a hydrogel with cellulose or polyacrylate derivatives or other suitable viscosity modifiers; a dry powder; aerosol with butane, propane, HFA, or CFC propellants; a dressing, such as, a tulle dressing, with white soft paraffin or polyethylene glycol impregnated gauze dressings or with hydrogel, hydrocolloid, or alginate film dressings. The compositions may also be administered intra-ocularly as an eye drop with appropriate buffers, viscosity modifiers (for example, cellulose derivatives), and preservatives (for example, benzalkonium chloride).

The pharmaceutical compositions of the invention may also be incorporated into a transdermal patch comprising nalmefene. Details of such patches can be found in, for example, WO2017/125455 and U.S. Pat. No. 6,569,866, the details of which are incorporated by reference herein.

For oral administration, capsules, boluses, or tablets may be prepared by mixing the pharmaceutical compositions of the invention with a suitable finely divided diluent or carrier, additionally containing a disintegrating agent and/or binder such as starch, lactose, talc, or magnesium stearate.

For parenteral administration injectable formulations may be prepared in the form of a sterile solution or emulsion.

The compositions of the present invention may be presented in unit dosage form and may be prepared by any of the methods well known in the art of pharmacy. The term “unit dosage form” means a single dose wherein all active and inactive ingredients are combined in a suitable system, such that the patient or person administering the drug can open a single container or package with the entire dose contained therein, and does not have to mix any components together from two or more containers or packages. Typical examples of unit dosage forms are tablets or capsules for oral administration or transdermal patches comprising the unit dosage. These examples of unit dosage forms are not intended to be limiting in any way, but merely to represent typical examples in the pharmacy arts of unit dosage forms.

In one aspect the invention provides unit dosage forms comprising about 0.01 to about 10 mg nalmefene and at least one pharmaceutically acceptable carrier or excipient. In one embodiment a unit dosage form comprises about 0.01 to about 2.0 mg of nalmefene and at least one pharmaceutically acceptable carrier or excipient. In one embodiment a unit dosage form comprises about 0.05 to about 0.6 mg nalmefene and at least one pharmaceutically acceptable carrier or excipient.

In one aspect the invention provides unit dosage forms comprising about 0.01 to about 10 mg nalmefene, about 0.05 to about 5 mg, about 0.1 to about 1 mg, about 0.2 to about 0.9 mg, about 0.3 to about 0.8 mg, about 0.4 to about 0.7, about 0.5 mg to about 0.6 mg, or about 0.6 mg nalmefene, and at least one pharmaceutically acceptable carrier or excipient.

In one embodiment a unit dosage form comprises less than about 1 mg, preferably less than about 0.7 mg nalmefene, preferably about 0.6 mg nalmefene.

In another embodiment, the unit dosage form is for treating a demyelinating disease in a subject in need thereof, preferably wherein the subject has MS. In another embodiment, the unit dosage is formulated for treating a demyelinating disease in a subject in need thereof. In one embodiment the demyelinating disease is MS.

In another embodiment the unit dose is formulated for increasing remyelination in a subject in need thereof, preferably wherein the subject has MS.

In one embodiment, the unit dosage form is for oral administration, preferably the unit dosage form is formulated for oral administration. In another embodiment, the unit dosage form is a transdermal patch.

The term “about” as used herein means a reasonable amount of deviation of the modified term such that the end result is not significantly changed. For example, when applied to a value, the term should be construed as including a deviation of +/−5% of the value.

Pharmaceutical compositions of nalmefene can be used in combination with other therapies for treating demyelination diseases.

### 6.3 Therapeutic Uses of Nalmefene

The inventors have surprisingly found that nalmefene gives rise to many positive effects in demyelination and MS mouse models. For example, the inventors have found that nalmefene is effective at treating demyelination in mouse models of EAE and cuprizone-induced demyelination, results that are translatable to treating demyelinating diseases such as MS in humans. The inventors have also found that nalmefene is unexpectedly effective at increasing remyelination in subjects in need thereof. Accordingly, this drug, which has a proven safety record, could be highly beneficial in the treatment of demyelination diseases and/or for increasing remyelination.

As set out in Examples 1 and 7, nalmefene promotes functional recovery from EAE-induced paralysis. Nalmefene also reduces EAE-induced total disability, with some doses showing a 70% reduction in disease (see Example 2) and promotes recovery from EAE-induced weight loss (see Example 3).

Nalmefene reduces immune cell infiltration into the brain in the EAE model of MS (see Examples 4 and 11). When administered before onset, nalmefene promotes functional recovery from paralysis, in the EAE model of MS (see Example 5).

Without wishing to be bound by theory, the inventors believe that nalmefene exerts its effects by increasing remyelination and/or decreasing demyelination. In Example 6, TEM images of the spinal cords of EAE mice treated with nalmefene resemble those of the healthy control.

Examples 7 to 10 investigate the effects of nalmefene on EAE induced paralysis.

Example 12 demonstrates the remyelination effects of nalmefene.

In Example 13, nalmefene does not deplete the major immune cell populations in the periphery despite reducing immune cell infiltration into the brain. In example 14, nalmefene promotes a switch in T helper cells from effector to memory cells suggestive of immune response resolution.

In Examples 15 and 16, the KOR is required for the full effect of nalmefene but nalmefene is effective at reducing disease independently of the KOR suggesting the full mechanism by which nalmefene exerts its effects is more complex than KOR activation. Additionally, antagonizing the MOR with BFNA does not recapitulate the disease recovery or reduction and demonstrates that the MOP antagonist activity alone is not responsible for the effects of nalmefene.

In Examples 17 and 18, nalmefene is shown to provide an almost complete recovery from EAE induced paralysis as compared to a drug known to reduce demyelination, clemastine fumerate.

The positive effects of nalmefene on mice were particularly surprising at dosages of 0.003 to 0.3 mg/kg, which can be converted to an equivalent human dose using the Regan-Shaw equation (Reagan-Shaw S; Nihal M; Ahmad N: Dose translation from animal to human studies revisited, FASEB J. 2007, Oct. 17).

Alternatively, dosages of 0.003 to 0.3 mg/kg, which can be converted to an equivalent human dose using a Rat Alcohol consumption model as shown below.

| | Route of | ED₅₀ | Inhibition | |
|---|---|---|---|---|
| Mouse Model | Administration | (μg/kq) | (%) | Reference |
| Alcohol | SC | 0.36 (4 | 73% | |
| consumption | | days) | 67% | |
| (Wistarand | | 0.36 (26 | | |
| AA rat) | | days) | | |
| Alcohol | SC | 0.18 (6 | 75% | |
| consumption | | days) | | |
| (Wistarrat) | | | | |
| Alcohol | PO | 16.1 | 23% | |
| consumption | | | | |
| (AA rat) | | | | |

In vivo efficacy ED₅₀ 200-400 μg/kg by SC administration

Underlying assumption: 300 μg/kg as the rat ED₅₀ SC dose.

Converting Dosage for Alcohol Consumption vs EAE
1. Assumption: cession of alcohol addition is a biomarker (surrogate) for EAE
   a. Likelihood of an association is moderate
2. Rat dose for cession of alcohol consumption is 300 μg/kg/day SC
3. Mouse dose for EAE is 10 μg/kg IP
4. Ratio in alcohol (rat) to EAE (mouse)=30 (assuming SC is similar to IP)
5. Using the ratio of efficacy for alcohol consumption vs MS in rodent
6. Human dose for alcohol consumption=18 mg/body/day PO
7. Estimated MS (EAE) human dose is 0.6 mg/body/day PO

The skilled worker in the art appreciates that are alternative algorithms that can be used to convert an observed therapeutic dosage from a mouse model into an equivalent human dose once the effective mouse dosage has been demonstrated. Such algorithms may be used effectively by the skilled person to determine the appropriate human dose.

As many demyelinating diseases cause horribly debilitating symptoms, any improvement in treatment outcomes provides an important development.

The inventors have now identified that nalmefene is a surprisingly effective treatment for demyelinating diseases at low doses, in particular MS. Administration of an effective agent at low dose offers many benefits. In addition to the reduction of side-effects, low dose usage also reduces (a) the quantities of active pharmaceutical ingredients (APIs) released into the environment via excretion and (b) the amount of unwanted APIs requiring disposal.

Accordingly, in one aspect the invention provides a method of treating a demyelinating disease in a subject in need thereof, comprising administering a therapeutically effective amount of nalmefene to the subject.

In another aspect the invention provides a method of treating a demyelinating disease in a subject comprising identifying a subject who would benefit from a decreased level of demyelination and administering to the subject a therapeutically effective amount of an agent that decreases the level of demyelination in the subject relative to the level of demyelination before administering the agent, wherein the agent comprises nalmefene.

In another aspect the invention provides a method of treating a demyelinating disease in a subject in need thereof, comprising administering to the subject a therapeutically effective amount of an agent that decreases the level of demyelination in the subject relative to the level of demyelination before administering the agent, wherein the agent comprises nalmefene.

In another aspect the invention provides a method of increasing remyelination in a subject in need thereof, comprising administering a therapeutically effective amount of nalmefene to the subject.

In another aspect the invention provides a method of increasing remyelination in a subject comprising identifying a subject who would benefit from an increased level of remyelination and administering to the subject a therapeutically effective amount of an agent that increases the level of remyelination in the subject relative to the level of remyelination before administering the agent, wherein the agent comprises nalmefene.

In another aspect the invention provides a method of increasing remyelination in a subject in need thereof, comprising administering to the subject a therapeutically effective amount of an agent that increases the level of remyelination in the subject relative to the level of remyelination before administering the agent, wherein the agent comprises nalmefene.

The term “treating” as used herein with reference to a disease or condition refers to the following: (a) ameliorating the disease or condition such as by eliminating or causing regression of or decreasing the severity of the disease or medical condition of the subject being treated relative to an untreated subject according to art-accepted criteria for monitoring the disease or condition (Wattjes et al. (2015). Evidence-based guidelines: MAGNIMS consensus guidelines on the use of MRI in multiple sclerosis—establishing disease prognosis and monitoring patients. Nat. Rev. Neurol. 11, 597-606; Traboulsee et al. (2016). Revised Recommendations of the Consortium of MS Centers Task Force for a Standardized MRI Protocol and Clinical Guidelines for the Diagnosis and Follow-Up of Multiple Sclerosis. AJNR Am. J. Neuroradiol. 37, 394-401; Toosy et al. (2014). Optic neuritis. Lancet Neurol. 13, 83-99; Ontaneda et al. (2017). Clinical outcome measures for progressive MS trials. Mult. Scler. 23, 1627-1635; Naismith et al. (2012). Diffusion tensor imaging in acute optic neuropathies: predictor of clinical outcomes. Arch. Neurol. 69, 65-71); (b) suppressing the disease or condition such as by slowing or arresting the development of the disease or condition relative to an untreated subject according to art-accepted criteria for monitoring the disease or condition (Oh et al. (2019). Imaging outcome measures of neuroprotection and repair in MS: A consensus statement from NAIMS. Neurology; Sormani et al. (2017). Assessing Repair in Multiple Sclerosis: Outcomes for Phase II Clinical Trials. Neurother. J. Am. Soc. Exp. Neurother. 14, 924-933; Zhang et al. (2018). Clinical trials in multiple sclerosis: milestones. Ther. Adv. Neurol. Disord. 11; Bjartmar et al. (2003). Axonal loss in the pathology of MS: consequences for understanding the progressive phase of the disease. J. Neurol. Sci. 206, 165-171; Toosy et al. (2014). Optic neuritis. Lancet Neurol. 13, 83-99) or (c) alleviating a symptom of the disease or condition in the subject relative to an untreated subject according to art-accepted criteria for monitoring the disease or condition (van Munster et al. (2017). Outcome Measures in Clinical Trials for Multiple Sclerosis. CNS Drugs 31, 217-236; Uitdehaag (2018). Disability Outcome Measures in Phase III Clinical Trials in Multiple Sclerosis. CNS Drugs 32, 543-558; Toosy et al. (2014). Optic neuritis. Lancet Neurol. 13, 83-99). In some preferred embodiments “treating” refers to ameliorating as in (a), suppressing as in (b) and/or alleviating as in (c) in a statistically significant manner relative to an appropriate untreated control subject according to art-accepted criteria for monitoring the disease or condition.

In the definition of “treating” the art accepted criteria are one or more of Criteria for measuring disability may include the expanded disability scale, multiple sclerosis functional composite Z-score and multiple sclerosis Impact Scale and Medical Outcomes Study Short Form, imaging of the brain, spinal cord or optic nerve, Multiple Sclerosis Functional Composite, and novel composite measures of disability, in addition to tests evaluating manual dexterity, ambulation, vision (including measures of axial diffusivity, visual acuity, contrast sensitivity, visual evoked potentials (VEPs), and thickness of the retinal nerve fiber layer (RNFL) and cognition.

The subject may show an observable or measurable decrease in one or more of the symptoms associated with or related to the disease or condition as known to those skilled in the art, as indicating improvement. In some embodiments, the disease or condition is a demyelinating disease, preferably MS, and the subject shows an observable and measurable decrease in one or more of the symptoms associated with or related to MS, preferably a decrease in demyelination as known to those skilled in the art, as indicating improvement. In preferred embodiments the improvement is a statistically significant improvement relative to an appropriate untreated control subject according to art-accepted criteria for monitoring the disease or condition.

The terms “decrease” and “reduced” (and grammatical variations thereof) as used herein with reference to demyelination mean any measurable or observable reduction in an amount or level of demyelination or of any symptom of a demyelinating disease that is attributable to demyelination in a treated subject relative to the level of demyelination in an appropriate control (e.g., untreated) subject. In preferred embodiments the measurable or detectable decrease or reduction is a statistically significant decrease or reduction, relative to an appropriate control.

The term “increase” (and grammatical variations thereof as used herein with reference to demyelination means any measurable or observable increase in an amount or level of remyelination or an improvement of any symptom of a demyelinating disease that is attributable to remyelination in a treated subject relative to the level of remyelination in an appropriate control (e.g., untreated) subject; e.g., placebo or non-active agent. An example of quantifying remyelination is demonstrated with treatment with clemastine fumarate using measures of VEPs to evaluate remyelination and recovery. (Green et al. (2017) Clemastine fumarate as a remyelinating therapy for multiple sclerosis (ReBUILD): a randomised, controlled, double-blind, crossover trial. Lancet. 390, 2481-2489; Jankowska-Lech et al. (2019). Peripapillary retinal nerve fiber layer thickness measured by optical coherence tomography in different clinical subtypes of multiple sclerosis. Mult. Scler. Relat. Disord. 27, 260-268; Naismith et al. (2012). Diffusion tensor imaging in acute optic neuropathies: predictor of clinical outcomes. Arch. Neurol. 69, 65-71; Oh et al. (2019). Imaging outcome measures of neuroprotection and repair in MS: A consensus statement from NAIMS. Neurology; Sormani et al. (2017). Assessing Repair in Multiple Sclerosis: Outcomes for Phase II Clinical Trials. Neurother. J. Am. Soc. Exp. Neurother. 14, 924-933. In preferred embodiments the measurable or detectable reduction is a statistically significant reduction, relative to an appropriate control.

The terms “administration of” or “administering” should be understood to mean providing nalmefene or a pharmaceutical composition comprising, consisting essentially of, or consisting of, nalmefene to the subject in need of treatment in a therapeutically useful form for the mode of administration. Nalmefene can be administered via any suitable route. Potential routes of administration include without limitation oral, parenteral (including intramuscular, subcutaneous, intradermal, intravenous, intraarterial, intramedullary and intrathecal), intraperitoneal, and topical (including dermal/epicutaneous, transdermal, mucosal, transmucosal, intranasal (e.g., by nasal spray or drop), intraocular (e.g., by eye drop), pulmonary (e.g., by inhalation), buccal, sublingual, rectal and vaginal.

The term “therapeutically” as used herein means “at disease onset”.

In certain embodiments, nalmefene is administered via oral dosage forms such as tablets, capsules, syrups, suspensions, and the like. In another embodiment, nalmefene is administered via a transdermal patch.

The term “therapeutically effective amount” refers to a sufficient quantity of the active agent, in a suitable composition, and in a suitable dosage form to treat the noted disease conditions. The “therapeutically effective amount” will vary depending on the compound, the severity of the demyelination disease, and the species, age, weight, etc., of the subject to be treated.

Preferably, the therapeutically effective amount of nalmefene is the amount equivalent to about 0.003-about 0.3 mg/kg in a mouse, which can be converted according to accepted practice into an animal or human subject dosage. For example, using the Reagan-Shaw equation, a therapeutically effective amount of nalmefene for a dog would be about 0.67-about 2 mg/kg.

In one embodiment the therapeutically effective amount of nalmefene is the amount equivalent to about 0.003-about 0.3 mg/kg in a mouse, converted using the Rat Alcohol consumption model described herein.

In one embodiment the therapeutically effective amount of nalmefene to be administered to a human subject is about 0.01 to about 10 mg nalmefene to the subject daily, 0.01 to about 2.0 mg nalmefene to the subject daily, or about 0.05 to about 0.6 mg nalmefene to the subject daily.

In another embodiment the method comprises administering about 0.01 to about 10 mg nalmefene, about 0.05 to about 5 mg, about 0.1 to about 1 mg, about 0.2 to about 0.9 mg, about 0.3 to about 0.8 mg, about 0.4 to about 0.7, about 0.5 mg to about 0.6 mg, or about 0.6 mg nalmefene daily.

In one embodiment the method comprises a long duration therapy.

In some embodiments the long duration therapy comprises administration of a therapeutically effective dose of nalmefene to a subject in need thereof for at least 5 days, at least 6 days, or at least 7 days.

In some embodiments the long duration therapy comprises administration of a therapeutically effective dose of nalmefene to a subject in need thereof for at least 5, preferably at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, preferably at least 90 days.

In some embodiments a long duration therapy comprises administration of a therapeutically effective dose of nalmefene to a subject in need thereof for at least a week, at least 2 weeks, at least 3 weeks, at least 4 weeks, at least 6 weeks, or at least 8 weeks.

In some embodiments the long duration therapy comprises administration for at least 5 days, at least 6 days, at least 7 days, at least 14 days, for at least 21 days, for at least 28 days, for at least 35 days, for at least 42 days, for at least 45 days, for at least 60 days, for at least 120 days, for at least 240 days, or for at least 360 days.

In some embodiments a long duration therapy comprises administration of a therapeutically effective dose of nalmefene to a subject in need thereof for at least 1 week, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or at least 52 weeks.

In some embodiments a long duration therapy comprises administration of a therapeutically effective dose of nalmefene to a subject in need thereof for at least 1 month, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or at least 36 months.

In some embodiments the long duration therapy comprises a dosing gap, preferably wherein the dosing gap is at least 1 day.

In some embodiments dosing gap comprises at least 1, 2, 3, 4, 5, 6, 7, 8, 9, 10, 11, 12, 13, or 14 days.

In some embodiments the dosing gap comprises at least 1, 2, 3, 4, 5, 6, 7, 8, 9, 10, 11, or 12 weeks.

In some embodiments the dosing gap comprises at least 1, 2, 3, 4, 5, 6, 7, 8, 9, 10, or 11 months.

The term “demyelinating disease” refers to a disease of the nervous system in which the myelin sheath of neurons is damaged. Demyelinating diseases include demyelinating myelinoclastic diseases and demyelinating leukodystrophic diseases.

Demyelinating diseases may affect the central nervous system and peripheral nervous system. The central nervous system demyelinating diseases include multiple sclerosis, optic neuritis, Devic's disease, inflammatory demyelinating diseases, central nervous system neuropathies like those produced by Vitamin B12 deficiency, myelopathies like Tabes dorsalis, leukoencephalopathies like progressive multifocal leukoencephalopathy, leukodystrophies, or a combination thereof. The peripheral nervous system demyelinating diseases include Guillain-Barre syndrome and its chronic counterpart, chronic inflammatory demyelinating polyneuropathy, anti-MAG peripheral neuropathy, Charcot Marie Tooth (CMT) disease, copper deficiency, progressive inflammatory neuropathy, or a combination thereof. The term “subject” refers to a mammal, more preferably a human, or companion animal. Preferred companion animals include cats, dogs and horses. Other mammalian subjects include agricultural animals, including horses, pigs, sheep, goats, cows, deer, or fowl: and laboratory animal, including monkeys, rats, mice, rabbits and guinea pig.

The invention also provides a use of nalmefene in the manufacture of a medicament for treating a demyelinating disease in a subject in need thereof.

In another aspect the invention provides a method of attenuating demyelination in a subject in need thereof, comprising administering a therapeutically effective amount of nalmefene to the subject and thereby attenuating a level of demyelination in the subject relative to the level of demyelination when nalmefene is not administered.

In another aspect the invention provides a method of attenuating demyelination in a subject in need thereof, comprising administering a therapeutically effective amount of an agent that decreases the level of demyelination in the subject relative to the level of demyelination before administering the agent and/or that increases the level of remyelination in the subject relative to the level of remyelination before administering the agent wherein the agent comprises nalmefene.

In one embodiment the therapeutically effective amount of nalmefene to be administered to a human subject is about 0.01 to about 10 mg nalmefene to the subject daily, 0.01 to about 2.0 mg nalmefene to the subject daily, or about 0.05 to about 0.6 mg nalmefene to the subject daily.

In another embodiment the therapeutically effective amount of nalmefene to be administered to a human subject is about 0.01 to about 10 mg nalmefene, about 0.05 to about 5 mg, about 0.1 to about 1 mg, about 0.2 to about 0.9 mg, about 0.3 to about 0.8 mg, about 0.4 to about 0.7, about 0.5 mg to about 0.6 mg, or about 0.6 mg nalmefene daily.

The term “attenuation of demyelination” means in certain embodiments that the amount or level of demyelination in the subject as a result of the disease or as a symptom of the disease is reduced when compared to otherwise identical conditions in an appropriate control subject or at an appropriate control reference timepoint and/or in certain embodiments that the amount or level of remyelination in the subject is increased when compared to an otherwise identical conditions in an appropriate control subject or at an appropriate control reference timepoint. In some preferred embodiments the reduction or increase as compared to the appropriate control is a statistically significant reduction or increase.

In certain preferred embodiments, the term “attenuation of demyelination” thus means that the amount of or level demyelination in the subject as a result of the disease or as a symptom of the disease is reduced or decreased in a statistically significant manner when compared to a suitable control as would be understood by a person of skill in the art in view of the present disclosure and/or the amount or level of remyelination in the subject is increased in a statistically significant manner when compared to a suitable control as would be understood by a person of skill in the art in view of the present disclosure.

Similarly, the term “improvement in nerve function” refers to a quantifiable improvement in function having a statistically different change in a measurable parameter relative to an appropriate control as recognized by a person of skill in the art. In some embodiments the improvement in function has a statistically significant change in the measurable parameter. In one embodiment the measurable parameter is the disease score as described in Example 1.

Symptoms attributable to demyelination will vary depending on the disease but may include, for example but not limited to, neurological deficits, such as chronic pain, cognitive impairment (including memory, attention, conceptualization and problem-solving skills) and information processing; paresthesia in one or more extremities, in the trunk, or on one side of the face; weakness or clumsiness of a leg or hand; or visual disturbances, e.g. partial blindness and pain in one eye (retrobulbar optic neuritis), dimness of vision, or scotomas.

The invention also provides a use of nalmefene in the manufacture of a medicament for attenuating demyelination in a subject in need thereof.

The invention also provides nalmefene for use for attenuating demyelination in a subject in need thereof.

In another aspect the invention provides a method of treating MS in a subject in need thereof, comprising administering a therapeutically effective amount of nalmefene to the subject. The subject may suffer from any type of MS including CIS, RRMS, PRMS, SPMS, PRMS or MS that follows a different and/or undefined disease course.

In another aspect the invention provides a method of treating MS in a subject in need thereof, comprising administering to the subject a therapeutically effective amount of an agent that decreases a level of demyelination in the subject relative to the level before administering the agent and/or that increases a level of remyelination in the subject in the subject relative to the level before administering the agent, wherein the agent comprises nalmefene.

The invention also provides a use of nalmefene in the manufacture of a medicament for treating MS in a subject in need thereof.

The invention also provides nalmefene for use for treating MS in a subject in need thereof.

In some embodiments the methods of treating MS set forth herein can comprise one or more of the following steps selected from the group consisting of diagnosing MS in the subject, testing for demyelination in the subject, testing for a reduction or reversal in demyelination in the subject, testing for remyelination in the subject, testing for a level of paralysis or a reduction or reversal of a level of paralysis in the subject, and testing for a decrease or increase of coordination and/or balance in the subject.

In one embodiment a method of treating a demyelinating disease and/or of attenuating demyelination and/or of treating MS and/or of increasing remyelination comprises identifying a subject who would benefit from a level of decreased demyelination.

In some embodiments a subject who would benefit from a level of decreased demyelination is identified on the basis of exhibiting one or more clinical symptoms of MS including, but not limited to: loss of sensitivity or changes in sensation such as tingling, pins and needles or numbness, muscle weakness of variable severity, very pronounced reflexes, muscle spasms, or difficulty in moving; difficulties with coordination and balance (ataxia); spasticity; problems with speech or swallowing, visual problems (nystagmus, optic neuritis or double vision), fatigue, acute or chronic pain, facial pain (trigeminal neuralgia), bladder and bowel difficulties, incontinence, reduced cognitive ability, depression, anxiety and other emotional abnormalities, sexual dysfunction, Uhthoff's phenomenon (a worsening of symptoms due to exposure to higher than usual temperatures), and Lhermitte's sign (an electrical sensation that runs down the back when bending the neck).

In one embodiment the therapeutically effective amount of nalmefene to be administered to a human subject is about 0.01 to about 10 mg nalmefene to the subject daily, 0.01 to about 2.0 mg nalmefene to the subject daily, or about 0.05 to about 0.6 mg nalmefene to the subject daily.

In another embodiment the therapeutically effective amount of nalmefene to be administered to a human subject is about 0.01 to about 10 mg nalmefene, about 0.05 to about 5 mg, about 0.1 to about 1 mg, about 0.2 to about 0.9 mg, about 0.3 to about 0.8 mg, about 0.4 to about 0.7, about 0.5 mg to about 0.6 mg, or about 0.6 mg nalmefene daily.

In one embodiment the treatment results in one or more clinical outcomes as compared to subjects not treated with nalmefene selected from the group consisting of:
(a) a decrease in MS disease progression;
(b) a decrease in MS disease severity;
(c) a decrease in nerve cell demyelination;
(d) a decrease in frequency or severity of relapsing MS attacks;
(e) a decrease in MS clinical symptoms;
(f) the healing of damaged nerve tissue (neuro-restoration);
(g) an increase in remyelination of demyelinated nerves in the central nervous system (neuro-restoration/protection);
(h) the protection of damaged nerve tissue from further disease activity (neuroprotection);
(i) the promotion neuronal outgrowth (neuro-regeneration) in the central nervous system;
(j) a decrease in disability caused by MS;
(k) an improvement of nerve function; and
(l) an enhanced rate of remission.

In another embodiment the treatment results in a reduction of one or more clinical symptoms of MS including, but not limited to loss of sensitivity or changes in sensation such as tingling, pins and needles or numbness, muscle weakness of variable severity, very pronounced reflexes, muscle spasms, or difficulty in moving; difficulties with coordination and balance (ataxia); spasticity; problems with speech or swallowing, visual problems (nystagmus, optic neuritis or double vision), fatigue, acute or chronic pain, facial pain (trigeminal neuralgia), bladder and bowel difficulties, incontinence, reduced cognitive ability, depression, anxiety and other emotional abnormalities, sexual dysfunction, Uhthoff's phenomenon (a worsening of symptoms due to exposure to higher than usual temperatures), and Lhermitte's sign (an electrical sensation that runs down the back when bending the neck).

In one aspect the invention provides a method of accelerating remission of MS in a subject in need thereof, the method comprising administering a therapeutically effective amount of nalmefene to the subject.

In one aspect the invention provides a method of accelerating remission from MS in a subject in need thereof, the method comprising administering a therapeutically effective amount of an agent that decreases the level of demyelination in the subject relative to the level of demyelination before administering the agent, wherein the agent comprises nalmefene.

In one aspect the invention provides a method of accelerating remission from MS in a subject in need thereof, the method comprising administering a therapeutically effective amount of an agent that increases the level of remyelination in the subject relative to the level of remyelination before administering the agent, wherein the agent comprises nalmefene.

The invention also provides a use of nalmefene in the manufacture of a medicament for accelerating remission from MS in a subject in need thereof.

The invention also provides nalmefene for use for accelerating remission from MS in a subject in need thereof.

In some embodiments the therapeutically effective amount of nalmefene to be administered to a human subject is about 0.01 to about 10 mg nalmefene to the subject daily, 0.01 to about 2.0 mg nalmefene to the subject daily, or about 0.05 to about 0.6 mg nalmefene to the subject daily.

In another embodiment the therapeutically effective amount of nalmefene to be administered to a human subject is about 0.01 to about 10 mg nalmefene, about 0.05 to about 5 mg, about 0.1 to about 1 mg, about 0.2 to about 0.9 mg, about 0.3 to about 0.8 mg, about 0.4 to about 0.7, about 0.5 mg to about 0.6 mg, or about 0.6 mg nalmefene daily.

The term “enhanced rate of remission of MS” as used herein, means that the start of the remission process is reached faster and/or the rate at which remission is achieved is faster (as compared to subjects not treated with nalmefene).

Remission of MS can be measured using any technique known in the art including but not limited to physical disability status, biological markers and brain scans using MRI.

In one aspect the invention provides a method of treating MS in a human subject in need thereof, the method comprising administering to the subject about 0.01 to about 10 mg nalmefene to the subject daily, 0.01 to about 2.0 mg nalmefene to the subject daily, or about 0.05 to about 0.6 mg nalmefene to the subject daily, wherein the treatment results in one or more clinical outcomes as compared to subjects not treated with nalmefene selected from the group consisting of:
(a) a decrease in MS disease progression;
(b) a decrease in MS disease severity;
(c) a decrease in nerve cell demyelination;
(d) a decrease in frequency or severity of relapsing MS attacks;
(e) a decrease in MS clinical symptoms;
(f) the healing of damaged nerve tissue (neuro-restoration);
(g) an increase in remyelination of demyelinated nerves in the central nervous system (neuro-restoration/protection);
(h) the protection of damaged nerve tissue from further disease activity (neuroprotection);
(i) the promotion neuronal outgrowth (neuro-regeneration) in the central nervous system;
(j) a decrease in disability caused by MS;
(k) an improvement of nerve function; and
(l) an enhanced rate of remission.

In one aspect the invention provides a method of treating MS in a human subject in need thereof, the method comprising administering to the subject about 0.01 to about 10 mg nalmefene, about 0.05 to about 5 mg, about 0.1 to about 1 mg, about 0.2 to about 0.9 mg, about 0.3 to about 0.8 mg, about 0.4 to about 0.7, about 0.5 mg to about 0.6 mg, or about 0.6 mg nalmefene daily, wherein the treatment results in one or more clinical outcomes as compared to subjects not treated with nalmefene selected from the group consisting of:
(a) a decrease in MS disease progression;
(b) a decrease in MS disease severity;
(c) a decrease in nerve cell demyelination;
(d) a decrease in frequency or severity of relapsing MS attacks;
(e) a decrease in MS clinical symptoms;
(f) the healing of damaged nerve tissue (neuro-restoration);
(g) an increase in remyelination of demyelinated nerves in the central nervous system (neuro-restoration/protection);
(h) the protection of damaged nerve tissue from further disease activity (neuroprotection);
(i) the promotion neuronal outgrowth (neuro-regeneration) in the central nervous system;
(j) a decrease in disability caused by MS;
(k) an improvement of nerve function; and
(l) an enhanced rate of remission.

In another aspect the invention relates to a method of treating a demyelinating disease in a subject comprising identifying a subject who would benefit from a decreased level of demyelination and administering to the subject a therapeutically effective amount of an agent that decreases the level of demyelination relative to the level of demyelination before administering the agent, wherein the agent comprises nalmefene.

In another aspect the invention relates to a method of increasing remyelination in a subject comprising identifying a subject who would benefit from an increased level of remyelination and administering to the subject a therapeutically effective amount of an agent that increases the level of remyelination relative to the level of remyelination before administering the agent, wherein the agent comprises nalmefene.

Specifically contemplated as embodiments of the invention described herein relating to nalmefene for use in decreasing demyelination, attenuating demyelination, accelerating remission of MS, treating MS, treating a demyelinating disease and increasing remyelination are all of the embodiments of the invention set forth herein relating to the aspects of the invention that are methods of decreasing demyelination, attenuating demyelination, accelerating remission of MS, treating MS, treating a demyelinating disease and increasing remyelination.

Additionally, specifically contemplated as embodiments of the invention described herein relating to the use of nalmefene in the manufacture of a medicament for decreasing demyelination, attenuating demyelination, accelerating remission of MS, treating MS or for increasing remyelination are all of the embodiments of the invention set forth herein relating to the aspects of the invention that are methods of decreasing demyelination, attenuating demyelination, accelerating remission of MS, treating MS, treating a demyelinating disease and increasing remyelination.

In addition, specifically contemplated herein for all recited method, use and nalmefene for use aspects of the invention are all of the embodiments set out herein that relate to long duration therapy and dosing gaps in long duration therapy.

The invention consists in the foregoing and also envisages constructions of which the following gives examples only and in no way limit the scope thereof.

### 6.4 Examples

### Example 1: Nalmefene Promotes Functional Recovery from Paralysis when Administered Therapeutically in the Experimental Autoimmune Encephalomyelitis (EAE) Model of MS

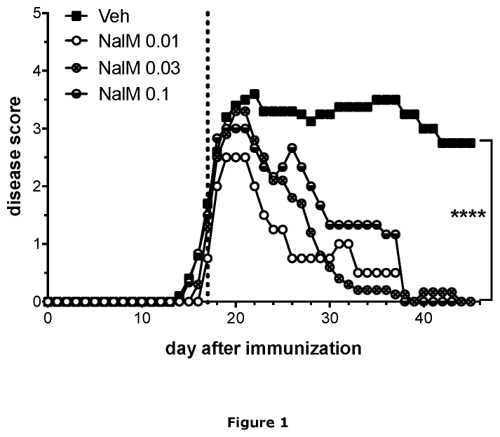

Experimental detail: Female, C57BL/6 mice were immunized subcutaneously (s.c.) in the hind flanks to induce EAE using myelin oligodendrocyte glycoprotein (MOG) peptide 35-55 (50 μg/mouse) in complete Freund's adjuvant containing heat-killed Mycobacterium tuberculosis (500 μg/mouse). In addition, pertussis toxin (200 ng/mouse) was administered intraperitoneally (i.p.) on days 0 and 2. Mice were weighed and scored daily. On day 17 (dotted line), mice were started on daily treatment with vehicle only (Veh; 10% tween and 10% DMSO in saline) or Nalmefene (NaIM) at 0.1, 0.03, or 0.01 mg/kg by i.p. injection. Nalmefene was supplied by University of Kansas, Synthetic Chemical Biology Core Laboratory, 97.6% pure by HPLC). Treatment allocation was blinded. The disease was scored from 0-5 with 0 (normal), 1 (partial tail paralysis), 2 (full tail paralysis), 3 (one hind limb paralysed or severe disability in both hind limbs), 4 (complete paralysis of both hind limbs) and 5 (moribund). This model is a standard disease model for multiple sclerosis and is described in White et al. 2018. Scientific Reports. 8:259 which is incorporated herein by reference in its entirety. Shown are results combined from 2 independent experiments. ****p<0.0001 by one-way ANOVA with Dunnett's multiple comparison test. Results are shown in FIG. 1.

Interpretation and impact: The results shown in FIG. 1 demonstrate that nalmefene is able to treat on-going disease. The reduction of disease in all nalmefene-treated groups indicates recovery from paralysis, which is unusual in this model. Finally, the doses at which nalmefene show the most rapid recovery are 0.03 and 0.01 mg/kg with doses above this level appearing less effective.

### Example 2: Nalmefene Reduces Total Disability when Administered Therapeutically in the EAE Model of MS

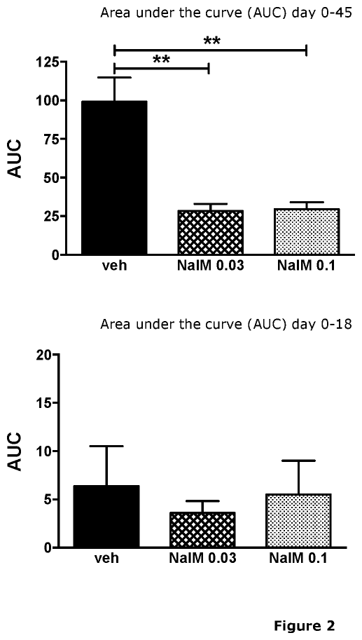

Experimental detail: EAE was induced in female C57BL/6 mice as described in Example 1. Results are shown in FIG. 2. On day 17, mice were started on daily treatment with vehicle only (Veh) or nalmefene (NaIM) at 0.1 or 0.03 mg/kg by i.p. injection. The area under the curve (AUC) was calculated for each mouse based upon the daily disease score and represents the total disability experienced. Shown are results from 1 representative experiment. *p<0.05 by one-way ANOVA with Dunnett's multiple comparison test.

Interpretation and impact: Despite all treatment groups having similar disease scores at the start of treatment (lower graph), mice treated daily with nalmefene had significantly lower total disability by day 45 after immunization to induce EAE (upper graph). Doses of 0.1 and 0.03 mg/kg nalmefene reduced disability similarly, equating to a 70% reduction in disease.

Without wishing to be bound by theory, the inventors believe that the results in Example 2 highlight the benefits of treatment with nalmefene over a period of at least a week. Accordingly, in some embodiment's administration comprises administration for at least 7 days, at least 14 days, at least 30 days, at least 45 days, at least 60 days, at least 120 days, at least 240 days, or at least 360 days.

### Example 3: Nalmefene Promotes Recovery from EAE-Induced Weight Loss when Administered Therapeutically

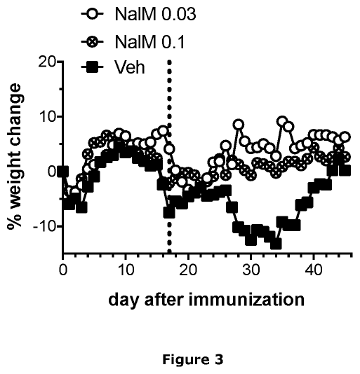

Experimental detail: EAE was induced in female C57BL/6 mice as described in Example 1. Results are shown in FIG. 3. Mice were weighed daily and the % change in body weight calculated. On day 17 (dotted line), mice were started on daily treatment with vehicle only (Veh) or nalmefene (NaIM) at 0.1 or 0.03 mg/kg by i.p. injection.

Interpretation and impact: At onset of disease, mice rapidly lose weight. Once treatment with nalmefene is initiated (dotted line), mice recover from EAE-induced weight loss.

### Example 4: Nalmefene Reduces the Immune Cell Infiltration into the Brain when Administered at Low Doses Therapeutically in the EAE Model of MS

Experimental detail: EAE was induced in female C57BL/6 mice as described in Example 1.

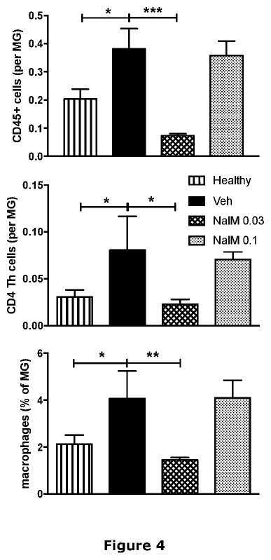

Results are shown in FIG. 4. On day 17, mice were started on daily treatment with vehicle only (Veh) or nalmefene (NaIM) at 0.1 or 0.03 mg/kg by i.p. injection. On day 45 after immunization to induce EAE, mice were culled and immune cells isolated from the brains. Isolation was by Percoll gradient as described in White et al. 2018. Scientific Reports. 8:259. Once isolated, cells were stained with fluorescently labelled antibodies to identify specific immune cell types and analysed by flow cytometry. All infiltrating immune cells were identified by CD45^{high} expression; CD4 T cells were identified as CD45^{high}CD4⁺, and macrophages as CD45^{high}CD11b⁺Gr-1⁻. The relative number of cells is expressed as a ratio to microglia (MG), a brain resident immune cell identified as CD45^{medium}CD11b⁺. ***p<0.001, **p<0.01, and *p<0.05 by one-way ANOVA with Dunnett's multiple comparison test.

Interpretation and impact: At day 45, there was a significant elevation in immune cells in the brains of vehicle-treated EAE mice compared to healthy animals. Treatment with 0.03 mg/kg nalmefene significantly reduced the number of infiltrating immune cells suggesting that at this dose, nalmefene may have immunomodulatory properties. Interestingly, while mice treated with 0.1 nalmefene had similar levels of infiltrating cells as vehicle-treated animals, these mice had no overt signs of disease and had recovered fully from paralysis (FIG. 1).

### Example 5: Nalmefene Promotes Functional Recovery from Paralysis when Administered Before the Onset of Paralysis in the EAE Model of MS

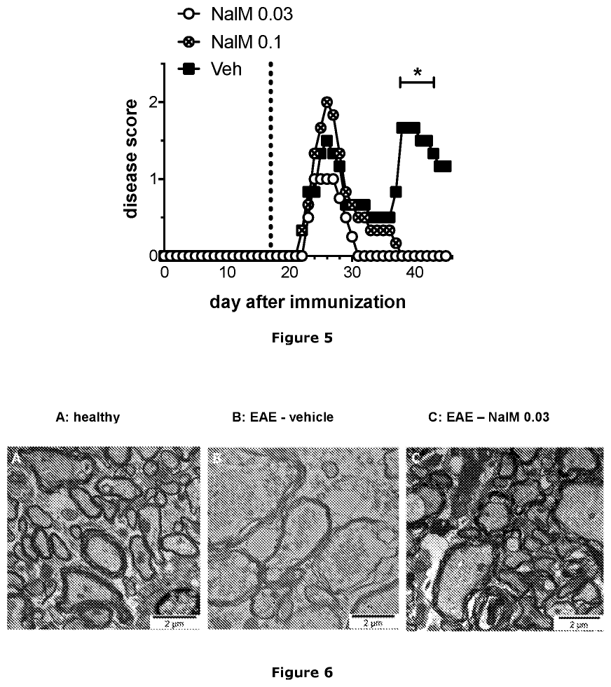

Experimental detail: EAE was induced in female C57BL/6 mice as described in Example 1. Results are shown in FIG. 5. On day 17 (dotted line), mice were started on daily treatment with vehicle only (Veh) or nalmefene (NaIM) at 0.1 or 0.03 mg/kg by i.p. injection. Shown are mice that were not sick at the time of treatment but developed disease later. *p<0.05 by two-way ANOVA with Holm-Sidak's multiple comparison test.

Interpretation and impact: Treating with nalmefene prior to disease onset did not alter the onset of disease. However, treatment with nalmefene led to a rapid recovery from paralysis compared to vehicle-treated mice. These data suggest that treating with nalmefene will also be effective at reducing total disability if administered before disease but may not prevent onset.

### Example 6: Myelination is Improved in Mice Treated with Nalmefene after the Onset of Paralysis in the EAE Model of MS

Experimental detail: EAE was induced in female C57BL/6 mice as described in Example 1. Results are shown in FIG. 6. On day 17, mice were started on daily treatment with vehicle only or nalmefene at 0.03 mg/kg by i.p. injection. On day 45 after immunization to induce EAE, mice were culled and spinal cords were processed for transmission electron microscopy (TEM). Shown are representative TEM images of spinal cord sections from a healthy (A), vehicle-treated EAE (B), or nalmefene-treated (C) EAE mouse stained to show that dark myelin rings around the nerve axons.

Interpretation and impact: At day 45, there was a significant reduction in the dark stained myelin in the spinal cord of the vehicle-treated EAE mice suggesting demyelination has occurred. Additionally, the nerve axons appear bloated and the cytoplasm disorganized suggesting cellular stress. In contrast, the nerve axons appear healthy and well myelinated in the nalmefene-treated mouse, which is concordant with full functional recovery.

### Example 7: Nalmefene Promotes Functional Recovery from Paralysis when Administered Therapeutically in the EAE Model of MS

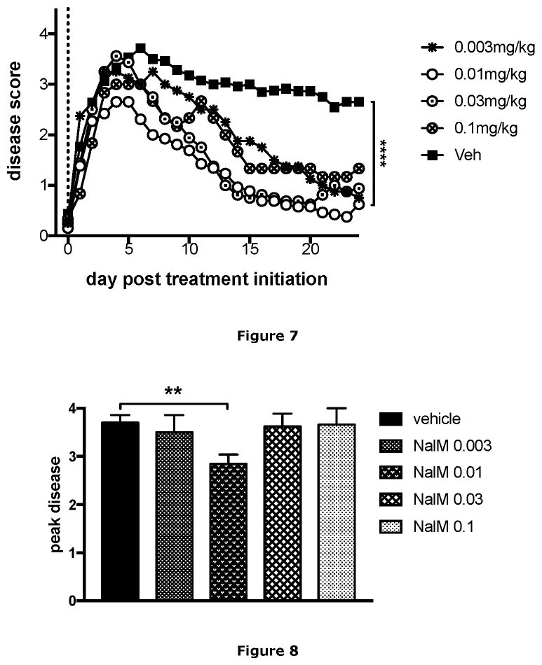

Experimental detail: EAE was induced in female C57BL/6 mice as described in Example 1. Results are shown in FIG. 7. On the day of disease onset (score >1, dotted line), mice were started on daily treatment with vehicle only (Veh) or nalmefene (NaIM) at 0.1, 0.03, 0.01, or 0.003 mg/kg by i.p. injection. Treatment allocation was blinded. Shown are the aligned scores from mice (n=15 in Veh, 3 in 0.1, 8 in 0.03, 13 in 0.01, and 4 in 0.003 mg/kg groups) starting from onset/treatment initiation. ****p<0.0001 by two-way ANOVA all doses compared to vehicle.

Interpretation and impact: By treating after the onset of disease, we show that nalmefene is able to treat on-going disease. The reduction of disease in all nalmefene-treated groups indicates recovery from paralysis, which is complete at 0.01 mg/kg and unusual in this model. Finally, the dose at which nalmefene shows the most rapid recovery is 0.01 mg/kg, and this finding has been replicated in 6 independent experiments.

### Example 8: Nalmefene Modestly Alters Peak Disease when Administered Therapeutically in the EAE Model of MS

Experimental detail: EAE was induced in female C57BL/6 mice as described in Example 1. Results are shown in FIG. 8. On the day of disease onset (score >1, dotted line), mice were started on daily treatment with vehicle only (Veh) or nalmefene (NaIM) at 0.1, 0.03, 0.01, or 0.003 mg/kg by i.p. injection. The peak disease score during the first EAE episode was recorded and shown are the mean and standard error of individual mice (n=15 in Veh, 3 in 0.1, 8 in 0.03, 13 in 0.01, and 4 in 0.003 mg/kg groups). **p<0.01 compared to vehicle by Kruskal-Wallis with Dunn's multiple comparison test.

Interpretation and impact: Because a difference in peak disease score was found at a dose of 0.01 mg/kg nalmefene compared to vehicle, nalmefene appears have a modest but significant effect on the initial immune-mediated neuroinflammatory event that leads to demyelination and paralysis. This finding indicates that the functional improvement observed (i.e. the recovery from paralysis) may occur because the initial insult has been repaired but also, in part, because the initial insult itself was reduced.

### Example 9: Nalmefene Promotes Full Recovery from EAE-Induced Paralysis when Administered Therapeutically

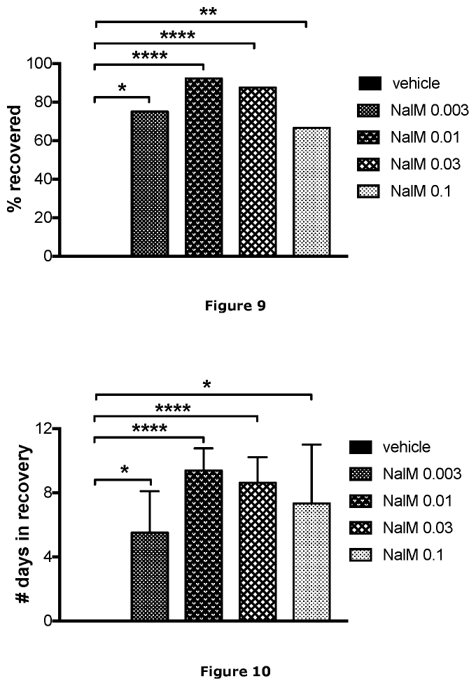

Experimental detail: EAE was induced in female C57BL/6 mice as described in Example 1. Results are shown in FIG. 9. On the day of disease onset (score >1, dotted line), mice were started on daily treatment with vehicle only or nalmefene at 0.1, 0.03, 0.01, or 0.003 mg/kg by i.p. injection. Mice were considered recovered if they received a score <0.5 by day 23 post treatment initiation. Shown are the percentages of mice in each group that recovered (n=15 in Veh, 3 in 0.1, 8 in 0.03, 13 in 0.01, and 4 in 0.003 mg/kg groups). ****p<0.0001, **p<0.01, and *p<0.05 by Fisher's exact test compared to vehicle. Interpretation and impact: Treatment with nalmefene enables full functional recovery (i.e. no paralysis) when administered therapeutically and at a wide range of doses (0.003-0.1 mg/kg all show a significant effect).

### Example 10: Nalmefene Promotes Sustained Recovery from EAE-Induced Paralysis when Administered Therapeutically

Experimental detail: EAE was induced in female C57BL/6 mice as described in Example 1. Results are shown in FIG. 10. On the day of disease onset (score >1), mice were started on daily treatment with vehicle only or nalmefene at 0.1, 0.03, 0.01, or 0.003 mg/kg by i.p. injection. Mice were considered recovered if they received a score <0.5 by day 23 post treatment initiation. Shown are the number of days mice were in recovery in each group (n=15 in Veh, 3 in 0.1, 8 in 0.03, 13 in 0.01, and 4 in 0.003 mg/kg groups). ****p<0.0001 and *p<0.05 by one-way ANOVA with Holm-Sidak's multiple comparison test.

Interpretation and impact: Treatment with nalmefene enables a sustained functional recovery (i.e. no paralysis) when administered therapeutically and at a wide range of doses (0.003-0.1 mg/kg all show a significant effect).

### Example 11: Nalmefene Reduces the Immune Cell Infiltration into the Brain when Administered Therapeutically in the EAE Model of MS

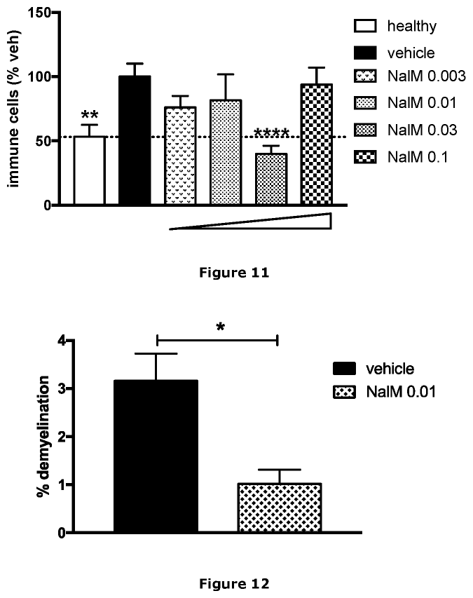

Experimental detail: EAE was induced in female C57BL/6 mice as described in Example 1. Results are shown in FIG. 11. On the day of disease onset (score >1), mice were started on daily treatment with vehicle only or nalmefene at 0.1, 0.03, 0.01, or 0.003 mg/kg by i.p. injection. During the chronic phase (>24 days post treatment initiation), mice were culled and immune cells isolated from the brains. Isolation was by Percoll gradient as described in White et al. 2018. Scientific Reports. 8:259. Once isolated, cells were stained with fluorescently labelled antibodies to identify specific immune cell types and analysed by flow cytometry. All infiltrating immune cells were identified by CD45^{high} expression. The relative number of cells is expressed as a ratio to microglia (MG), a brain resident immune cell identified as CD45^{medium}CD11b+. ****p<0.0001 and **p<0.01 by one-way ANOVA with Holm-Sidak's multiple comparison test compared to vehicle.

Interpretation and impact: In the chronic stage of EAE, there was a significant elevation in immune cells in the brains of vehicle-treated EAE mice compared to healthy animals. Treatment with 0.03 mg/kg nalmefene significantly reduced the number of infiltrating immune cells suggesting that this dose of nalmefene may have immunomodulatory properties. Interestingly, while mice treated with 0.1, 0.01, and 0.003 nalmefene had similar levels of infiltrating cells as vehicle-treated animals, these mice had no overt signs of disease and had recovered from paralysis (FIG. 7).

### Example 12: Myelination is Improved in Mice Treated with Nalmefene after the Onset of Paralysis in the EAE Model of MS

Experimental detail: EAE was induced in female C57BL/6 mice as described in Example 1. Results are shown in FIG. 12. On the day of disease onset (score >1), mice were started on daily treatment with vehicle only or nalmefene at 0.01 mg/kg by i.p. injection. During the chronic phase (>24 days post treatment initiation), mice were culled, perfused with 4% paraformaldehyde and spinal cords were processed for histology. Sections were stained with Luxel fast blue to assess the % area of the spinal cord that is demyelinated (i.e. does not stain with luxol fast blue). % demyelination was assessed using ImageJ. Shown are the means and standard error of individual values from vehicle (n=7) or 0.01 (n=3) nalmefene-treated EAE mice. *p<0.05 by one-way ANOVA with Holm-Sidak's multiple comparison test.

Interpretation and impact: During the chronic phase, when nalmefene enabled full functional recovery in mice, there was a significant reduction demyelination in the spinal cords of the nalmefene-treated EAE mice suggesting remyelination occurred.

### Example 13: Nalmefene does not Alter the Proportion of Major Lymphocyte Populations in the Spleen During the Chronic Phase of EAE

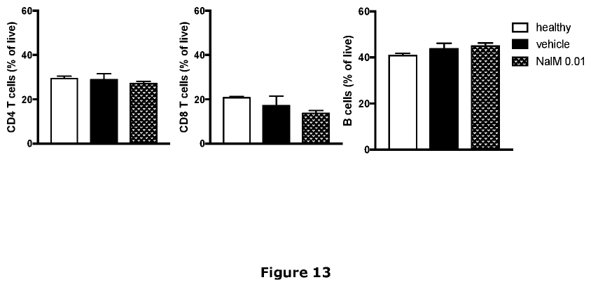

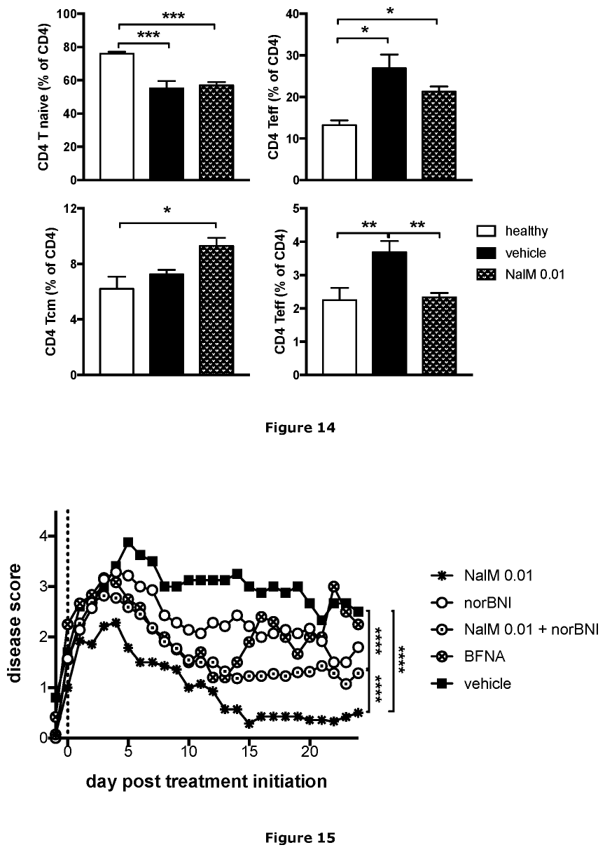

Experimental detail: EAE was induced in female C57BL/6 mice as described in Example 1. Results are shown in FIG. 13. On the day of disease onset (score >1), mice were started on daily treatment with vehicle only or nalmefene at 0.01 mg/kg by i.p. injection. During the chronic phase (27 days post treatment initiation), mice were culled and their splenocytes assessed by flow cytometry. The percentage of the major lymphocyte populations were identified using CD4 (CD4 T helper cells), CD8 (CD8 cytotoxic T cells), and B220 (B cells), and expressed as % live leukocytes (i.e. CD45⁺ cells). Shown are the means and standard error of individual mice with n=3 (healthy), 4 (vehicle) and 9 (NaIM). No significant differences were found between vehicle and healthy or nalmefene by one-way ANOVA with Holm-Sidak's multiple comparison test.

Interpretation and impact: Nalmefene does not alter the proportion of the major lymphocyte populations in the spleen despite reducing the number of infiltrating immune cells into the central nervous system. The maintenance of normal lymphocyte numbers in the spleen in the nalmefene-treated mice indicates that nalmefene does not reduce immune cell infiltration into the brain by killing immune cells.

### Example 14: Nalmefene does not Alter the Number of CD4 T Helper Cells in the Spleen, but Rather Shifts the CD4 T Cells from an Effector to Memory Phenotype Suggestive of Immune Resolution During the Chronic Phase of EAE

Experimental detail: EAE was induced in female C57BL/6 mice as described in Figure A. On the day of disease onset (score >1), mice were started on daily treatment with vehicle only or nalmefene at 0.01 mg/kg by i.p. injection. During the chronic phase (27 days post treatment initiation), mice were culled and their splenocytes assessed by flow cytometry. Naïve CD4 T cells (CD4⁺CD44⁻CD62L^{high}), effector CD4 T cells (CD4⁺CD44⁺CD62L⁻), and central memory CD4T cells (CD4⁺CD44⁺CD62L^{high}) are expressed as % CD4 T cells. “Teff:cm ratio” is the ratio of effector to central memory T cells. Shown are the means and standard error of individual mice with n=3 (healthy), 4 (vehicle) and 9 (NaIM). ***p<0.001, **p<0.01 and *p<0.05 by one-way ANOVA with Holm-Sidak's multiple comparison test.

Interpretation and impact: The increased effector to central memory ratio in the vehicle-treated mice with EAE compared to healthy mice indicates an on-going and active immune response mediated by CD4 T cells. The reduced ratio in the nalmefene-treated compared to the vehicle-treated mice indicates a shift toward a memory phenotype which occurs during the resolution phase of the immune response. The shift to a memory state indicates that immune resolution is occurring in nalmefene-treated mice in a model of MS where disease is driven by an active immune response.

### Example 15: Nalmefene Reduces Disease but does not Enable Full Recovery when the Kappa Opioid Receptor (KOR) is Blocked. Additionally, Blocking the Mu Opioid Receptor (MOR) with BFNA, a MOR Antagonist, does not Enable Significant and Sustained Recovery from Paralysis

Experimental detail: EAE was induced in female C57BL/6 mice as described in Example 1. Results are shown in FIG. 15. On the day of disease onset (score >1, dotted line), mice were treated with vehicle only (daily), nalmefene (0.01 mg/kg by i.p. injection daily), the KOR antagonist norBNI (10 mg/kg by i.p. injection weekly), both nalmefene and norBNI, or the MOR antagonist BFNA (10 mg/kg by i.p. injection weekly). Shown are the aligned scores from mice (n=7-9/group) starting from onset/treatment initiation. ****p<0.0001 by two-way ANOVA NaIM compared to vehicle or NalM+noBNI, and NalM+norBNI compared to vehicle.

Interpretation and impact: Administration of the KOR antagonist, norBNI, abolishes the ability of nalmefene to enable full recovery from paralysis (i.e. score <0.5), and this finding indicates that the KOR is required for the full effect of nalmefene. The finding that nalmefene is effective at reducing disease independently of the KOR (i.e. in the presence of norBNI) indicates that the full mechanism by which nalmefene exerts its effects is more complex than KOR activation. Because nalmefene is also a MOR antagonist, the finding that blocking the MOR with the MOR antagonist BFNA does not enable recovery in this model indicates that the MOR antagonistic activity alone is not involved in recovery, but KOR activation is.

### Example 16: Activation of the KOR is Required but Blocking the Activity of the MOR is not Required for Full Recovery from Paralysis Mediated by Nalmefene

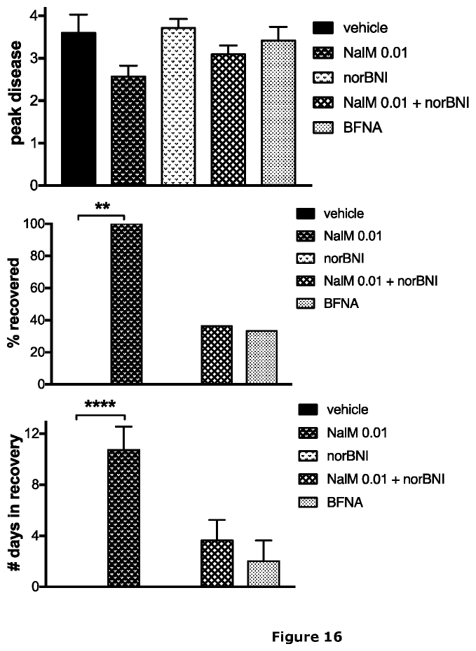

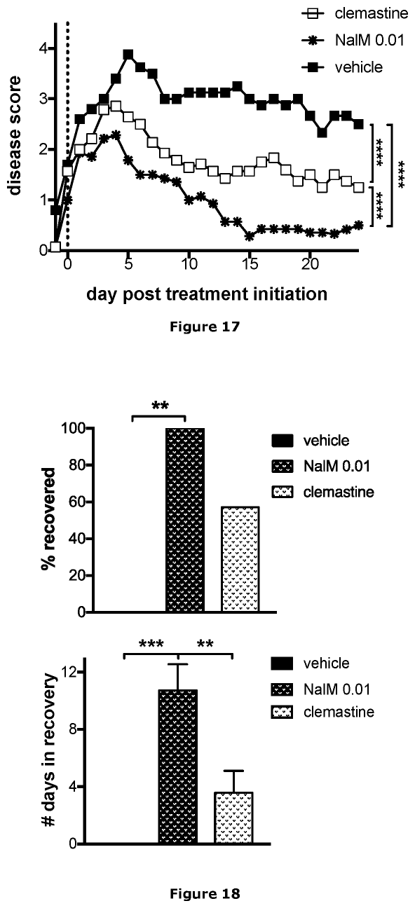

Experimental detail: EAE was induced in female C57BL/6 mice as described in Example 1. Results are shown in FIG. 16. On the day of disease onset (score >1, dotted line), mice were treated with vehicle only (daily), nalmefene (0.01 mg/kg by i.p. injection daily), the KOR antagonist norBNI (10 mg/kg by i.p. injection weekly), both nalmefene and norBNI, or the MOR antagonist BFNA (10 mg/kg by i.p. injection weekly). The peak disease score during the first EAE episode was recorded, and mice were considered recovered if they received a score <0.5 by day 23 post treatment initiation. Shown are the peak disease scores, the percentage of mice in each group that recovered, and the number of days in recovery (n=7-9/group). **p<0.01 and ****p<0.0001 by Fisher's exact test (% recovered) or one-way ANOVA with Holm-Sidak's multiple comparison test (#days in recovery).

Interpretation and impact: Administration of the KOR antagonist, norBNI, abolishes the ability of nalmefene to enable and sustain recovery from paralysis (i.e. score <0.5), but blocking the MOR with BFNA does not. This finding indicates that the KOR is required for the full effect of nalmefene at promoting full recovery, but the MOR is not.

### Example 17: Nalmefene is More Effective at Promoting Functional Recovery than Clemastine Fumerate, a Known Remyelinating Drug

Choice of clemastine fumerate: Previous research has identified the antihistamine, clemastine fumerate, in in vitro remyelination screens (Mei et al. Micropillar arrays as a high-throughput screening platform as a therapeutic in multiple sclerosis. Nat Med 2014; 20: 954-960). It has also been shown that clemastine fumerate is effective in the EAE and cuprizone models of MS. Clemastine fumarate is also the subject of an investigator-led clinical trial in MS patients with optic neuritis (Green A J et al. Clemastine fumarate as a remyelinating therapy for multiple sclerosis (ReBUILD): a randomised, controlled, double-blind, crossover trial. Lancet 2017; 390: 2481-2489). This clinical trial showed promising results with respect to remyelination by assessing visual function and highlighted that potential of repurposed compounds identified using current pre-clinical approaches.

Clemastine fumerate was as a positive control but also as a comparator to assess efficacy. In the original work described above, clemastine fumarate was used prophylactically (i.e. at the time of immunization) but still provided treated subjects with a similar reduction (but not ablation) of disease as we observed when we used clemastine fumerate therapeutically. In contrast to the reduction in disease mediated by clemastine fumerate, nalmefene led to almost complete recovery from paralysis.

Experimental detail: EAE was induced in female C57BL/6 mice as described in Figure A. On the day of disease onset (score >1, dotted line), mice were treated with vehicle only (daily, n=9), nalmefene (0.01 mg/kg by i.p. injection daily; n=7), or clemastine fumerate (10 mg/kg by i.p. injection; n=7). Shown are the aligned scores from mice starting from onset/treatment initiation. ****p<0.0001 by two-way ANOVA NaIM or clemastine compared to vehicle, and nalmefene compared to clemastine.

Interpretation and impact: Clemastine fumerate, an anti-histamine which also antagonizes the muscarinic receptor, has been shown to reduce chronic disability in the EAE model when used at 10 mg/kg starting at the time of immunization. Additionally, it has been shown to enhance remyelination in mice and humans [(Li et al. 2015, Clemastine rescues behavioral changes and enhances remyelination in the cuprizone mouse model of demyelination. Neurosci Bull.; 31: 617-625; Green, A. J et al., 2017 Clemastine fumarate as a remyelinating therapy for multiple sclerosis (ReBUILD): a randomised, controlled, double-blind, crossover trial. Lancet Lond. Engl. 390, 2481-2489]. In our EAE model, clemastine is similarly effective to previously published reports [Mei, F. et al. 2016, Accelerated remyelination during inflammatory demyelination prevents axonal loss and improves functional recovery. ELife 5] but is much less effective that nalmefene at enabling full functional recovery. This surprising finding indicates that nalmefene is superior to clemastine fumerate in this model.

### Example 18: Nalmefene Promotes a Greater and More Sustained Recovery than Clemastine Fumerate, a Known Remyelinating Drug

Experimental detail: EAE was induced in female C57BL/6 mice as described in Example 1. The results are shown in FIG. 18. On the day of disease onset (score >1, dotted line), mice were treated with vehicle only (daily, n=9), nalmefene (0.01 mg/kg by i.p. injection daily; n=7), or clemastine fumerate (10 mg/kg by i.p. injection; n=7). Mice were considered recovered if they received a score <0.5 by day 23 post treatment initiation. Shown are the percentage of mice in each group that recovered and the number of days in recovery. ***p<0.001 and **p<0.01 by Fisher's exact test (% recovered) or one-way ANOVA with Holm-Sidak's multiple comparison test (#days in recovery).

Interpretation and impact: Clemastine fumerate, an anti-histamine which also antagonizes the muscarinic receptor, has been shown to reduce chronic disability in the EAE model when used at 10 mg/kg starting at the time of immunization. Additionally, it has been shown to enhance remyelination in mice and humans. In our EAE model, clemastine fumerate treatment promotes recovery in just over 50% of the mice but the recovery is not sustained. In contrast, all of the mice recover when treated with nalmefene and have a sustained recovery. This surprising finding indicates that nalmefene is superior to clemastine fumerate in this model and provides a more sustained improvement in every animal treated.

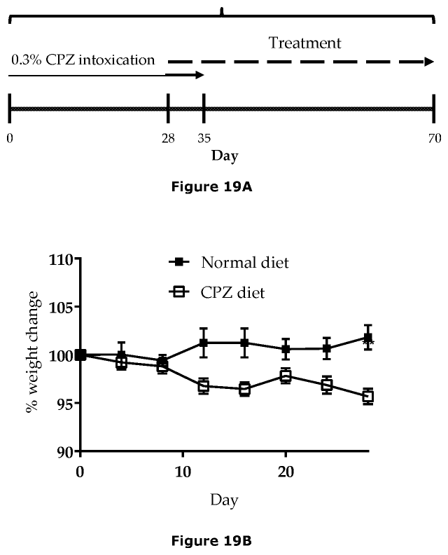

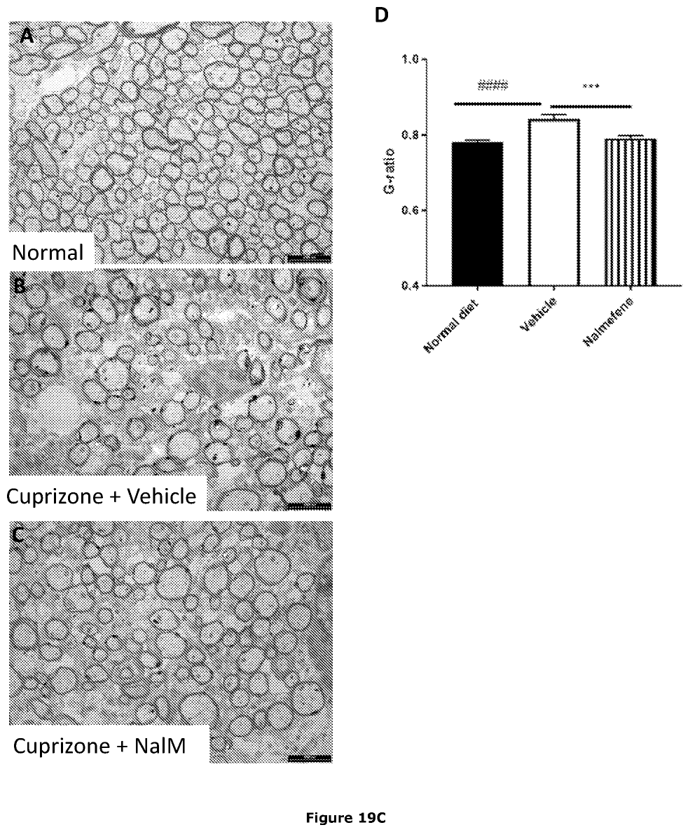

FIG. 19: Nalmefene Treatment Enhances Remyelination when Administered Therapeutically in the Cuprizone Demyelination Disease Model of MS.

FIG. 19A shows a time course of cuprizone induced demyelination and treatment regime.

Experimental details: A demyelinating disease state was induced in female C57BL/6 mice (8-14 weeks old and between 17-23 grams in weight). The mice were fed cuprizone-containing chow (0.3% (w/w) cuprizone) or chow only (normal controls) for 35 days, at which point they were switched back to standard chow. At day 28, mice were started on daily treatment with vehicle only (DMSO: Tween 80: Saline) or nalmefene at 0.01 mg/kg by i.p. injection. On day 70, mice were culled and brain tissue were processed for transmission electron microscopy (TEM). Mice were weighed daily and the % weight change calculated.

Interpretation and impact: This model is well established as a tool for the study of non-immune system induced demyelination. This model enables the assessment of putative remyelination-promoting therapeutics (Matsushima and Morell, 2001. The neurotoxicant, cuprizone, as a model to study demyelination and remyelination in the central nervous system. Brain Pathol. 11, 107-116).

FIG. 19B shows cuprizone induced weight change over the time course of study.

Experimental details: A demyelinating disease state was induced in female C57BL/6 mice as described in FIG. 19A.

Interpretation and impact: Mice treated with 0.3% cuprizone (CPZ) lose weight as the disease is induced, compared to mice with normal diet, corresponding to disease induction and severity.

FIG. 19C shows nalmefene treatment enhances remyelination when administered after demyelination in the cuprizone demyelination disease model of MS.

Experimental details: A demyelinating disease state was induced in female C57BL/6 mice as described in FIG. 19A. FIGS. 19A-C show representative Transmission Electron Microscopy (TEM) images of the corpus callosum of mice (A) fed normal diet and (B-C) fed 0.3% cuprizone to induce demyelination. Following the time course described in FIG. 19A, cuprizone fed mice were administered (B) vehicle only treatment, and (C) nalmefene (0.01 mg/kg/i.p.) and then sacrificed on experimental day 70. Scale bars represent 2000 nm.

FIG. 19 (D) shows the quantification and analysis of the g-ratios shows a significant difference between treatment groups F(3,953)=21.18 (p<0.0001). Mice fed a normal diet have a mean g-ratio of 0.78±0.09 in contrast to mice fed 0.3% cuprizone that have a significant increase in g-ratio of 0.84±0.1 corresponding to the decreased myelin thickness (####p<0.0001). Mice fed a diet with cuprizone treated with nalmefene (0.01 mg/kg/i.p.) (0.791±0.01) show a significant reduction in g-ratio compared to Vehicle treated controls (***p<0.001). Data represents measurements of 5 TEM images of the corpus callosum from 2-3 mice per treatment group and g-ratios calculated (a measure of myelin thickness) using Image J software. Analysis was performed by individuals blinded to treatment groups. (n=200-400 axons per treatment group). All data analysed by one-way ANOVA followed by Turkeys multiple comparisons test.

Interpretation and impact: As shown in the panels in FIG. 19, demyelination was very apparent in the corpus collosum of the brain of cuprizone-induced, vehicle only treated animals (B). The ratio between axonal circumference and myelin circumference (g-ratio) decreases with normal myelination. The cuprizone induced animals treated with nalmefene show a more normal axonal-myelin structure and organisation, and increased remyelination. Ultrastructurally, this nalmefene tissue is surprisingly similar to that of the naïve (normal) tissue. Quantitatively, the nalmefene tissue has a significantly lower g-ratio compared to vehicle only treated indicative of enhanced remyelination, with a g-ratio closer to that of naïve (normal) tissue. Qualitatively and quantitatively, nalmefene treatment enhances remyelination that is indicative of a near-full recovery following a demyelination insult of cuprizone.

1.-50. (canceled)

51. A method of treating a demyelinating disease in a subject in need thereof, the method comprising administering a pharmaceutical composition comprising nalmefene to the subject.

52. The method according to claim 51, wherein the pharmaceutical composition is administered to the subject for a period of (i) at least 7 days, (ii) at least 14 days, or (iii) at least one month.

53. The method according to claim 51, wherein the pharmaceutical composition is administered to the subject to obtain a dosage of about 0.01 to about 10 mg nalmefene daily.

54. The method according to claim 51, wherein the demyelinating disease is selected from the group consisting of multiple sclerosis (MS), optic neuritis, Devic's disease, inflammatory demyelinating diseases, central nervous system neuropathies, myelopathies, Tabes dorsalis, leukoencephalopathies, leukodystrophies, Guillain-Barre syndrome and its chronic counterpart, chronic inflammatory demyelinating polyneuropathy, anti-MAG (myelin-associated glycoprotein) peripheral neuropathy, Charcot Marie Tooth (CMT) disease, copper deficiency, progressive inflammatory neuropathy, and any combination thereof.

55. The method according to claim 51, wherein the demyelinating disease is multiple sclerosis (MS).

56. The method according to claim 54, wherein administration of the pharmaceutical composition to the subject results in one or more clinical outcomes selected from the group consisting of:
a decrease or delay in nerve cell demyelination;
a healing of damaged nerve tissue;
an increase in remyelination of demyelinated nerves in the subject's central nervous system;
neuroprotection;
protection of damaged nerve tissue from further disease activity;
promotion of neuronal outgrowth in the subject's central nervous system; and
an improvement in nerve function.

57. The method according to claim 54, wherein administration of the pharmaceutical composition to the subject results in one or more clinical outcomes selected from the group consisting of:
a decrease in demyelinating disease progression;
a decrease in demyelinating disease severity;
a decrease in frequency or severity of relapsing demyelinating disease attacks;
a decrease in disability caused by demyelinating disease;
a decrease in demyelinating disease clinical symptoms;
a decrease in paralysis;
an improvement in balance or coordination; and
an enhanced rate of remission.

58. The method according to claim 54, wherein administration of the pharmaceutical composition to the subject results in a reduction of one or more clinical symptoms selected from the group consisting of: loss of sensitivity, muscle weakness, impaired walking, impaired hand function, pronounced reflexes, muscle spasms, difficulty in moving, ataxia, spasticity, problems with speech or swallowing, visual problems, fatigue, acute or chronic pain, facial pain, incontinence, reduced cognitive ability, depression, anxiety, sexual dysfunction, Uhthoff s phenomenon, and Lhermitte's sign.

59. A method of increasing remyelination of nerves in a subject, the method comprising administering a pharmaceutical composition comprising nalmefene to the subject.

60. The method according to claim 59, wherein the pharmaceutical composition is administered to the subject for a period of (i) at least 7 days, (ii) at least 14 days, or (iii) at least one month.

61. The method according to claim 59, wherein the pharmaceutical composition is administered to the subject to obtain a dosage of about 0.01 to about 10 mg nalmefene daily

62. The method according to claim 59, wherein the subject has one or more symptoms of a demyelinating disease selected from the group consisting of multiple sclerosis (MS), optic neuritis, Devic's disease, inflammatory demyelinating diseases, central nervous system neuropathies, myelopathies, Tabes dorsalis, leukoencephalopathies, leukodystrophies, Guillain-Barre syndrome and its chronic counterpart, chronic inflammatory demyelinating polyneuropathy, anti-MAG (myelin-associated glycoprotein) peripheral neuropathy, Charcot Marie Tooth (CMT) disease, copper deficiency, progressive inflammatory neuropathy, and any combination thereof.

63. The method according to claim 59, wherein the subject has one or more symptoms of multiple sclerosis (MS).

64. The method according to claim 62, wherein administration of the pharmaceutical composition to the subject results in one or more clinical outcomes selected from the group consisting of:
a decrease or delay in nerve cell demyelination;
a healing of damaged nerve tissue;
an increase in remyelination of demyelinated nerves in the subject's central nervous system;
neuroprotection;
protection of damaged nerve tissue from further disease activity;
promotion of neuronal outgrowth in the subject's central nervous system; and
an improvement in nerve function.

65. The method according to claim 62, wherein administration of the pharmaceutical composition to the subject results in one or more clinical outcomes selected from the group consisting of:
a decrease in demyelinating disease progression;
a decrease in demyelinating disease severity;
a decrease in frequency or severity of relapsing demyelinating disease attacks;
a decrease in disability caused by demyelinating disease;
a decrease in demyelinating disease clinical symptoms;
a decrease in paralysis;
an improvement in balance or coordination; and
an enhanced rate of remission.

66. The method according to claim 62, wherein administration of the pharmaceutical composition to the subject results in a reduction of one or more clinical symptoms selected from the group consisting of: loss of sensitivity, muscle weakness, impaired walking, impaired hand function, pronounced reflexes, muscle spasms, difficulty in moving, ataxia, spasticity, problems with speech or swallowing, visual problems, fatigue, acute or chronic pain, facial pain, incontinence, reduced cognitive ability, depression, anxiety, sexual dysfunction, Uhthoff s phenomenon, and Lhermitte's sign.

67. A method of attenuating demyelination of nerves in a subject, the method comprising administering a pharmaceutical composition comprising nalmefene to the subject.

68. The method according to claim 67, wherein the pharmaceutical composition is administered to the subject for a period of (i) at least 7 days, (ii) at least 14 days, or (iii) at least one month.

69. The method according to claim 67, wherein the pharmaceutical composition is administered to the subject to obtain a dosage of about 0.01 to about 10 mg nalmefene daily.

70. The method according to claim 67, wherein the subject has one or more symptoms of a demyelinating disease selected from the group consisting of multiple sclerosis (MS), optic neuritis, Devic's disease, inflammatory demyelinating diseases, central nervous system neuropathies, myelopathies, Tabes dorsalis, leukoencephalopathies, leukodystrophies, Guillain-Barre syndrome and its chronic counterpart, chronic inflammatory demyelinating polyneuropathy, anti-MAG (myelin-associated glycoprotein) peripheral neuropathy, Charcot Marie Tooth (CMT) disease, copper deficiency, progressive inflammatory neuropathy, and any combination thereof.

71. The method according to claim 67, wherein the subject has one or more symptoms of multiple sclerosis (MS).

72. The method according to claim 70, wherein administration of the pharmaceutical composition to the subject results in one or more clinical outcomes selected from the group consisting of:
a decrease or delay in nerve cell demyelination;
a healing of damaged nerve tissue;
an increase in remyelination of demyelinated nerves in the subject's central nervous system;
neuroprotection;
protection of damaged nerve tissue from further disease activity;
promotion of neuronal outgrowth in the subject's central nervous system; and
an improvement in nerve function.

73. The method according to claim 70, wherein administration of the pharmaceutical composition to the subject results in one or more clinical outcomes selected from the group consisting of:
a decrease in demyelinating disease progression;
a decrease in demyelinating disease severity;
a decrease in frequency or severity of relapsing demyelinating disease attacks;
a decrease in disability caused by demyelinating disease;
a decrease in demyelinating disease clinical symptoms;
a decrease in paralysis;
an improvement in balance or coordination; and
an enhanced rate of remission.

74. The method according to claim 70, wherein administration of the pharmaceutical composition to the subject results in a reduction of one or more clinical symptoms selected from the group consisting of: loss of sensitivity, muscle weakness, impaired walking, impaired hand function, pronounced reflexes, muscle spasms, difficulty in moving, ataxia, spasticity, problems with speech or swallowing, visual problems, fatigue, acute or chronic pain, facial pain, incontinence, reduced cognitive ability, depression, anxiety, sexual dysfunction, Uhthoff s phenomenon, and Lhermitte's sign.
